(19) United States
(12) Patent Application Publication
(10) Pub. No.: US 2025/0078977 A1
(43) Pub. Date: Mar. 6, 2025

(54) METHOD AND SYSTEM FOR PROVIDING REMOTE PHYSIOTHERAPY SESSIONS

(71) Applicant: RAJIV TREHAN, BANGKOK (TH)

(72) Inventor: RAJIV TREHAN, BANGKOK (TH)

(21) Appl. No.: 18/241,919

(22) Filed: Sep. 4, 2023

(51) Int. Cl. G16H 20/30; A61B 5/00; A61B 5/11; G16H 30/40

(52) U.S. Cl. CPC G16H 20/30; A61B 5/0022; A61B 5/1128; A61B 5/4833; G16H 30/40; A61B 2505/09

(57) ABSTRACT

This disclosure relates to a method for providing remote physiotherapy sessions. The method includes capturing a first real-time video of a patient performing at least one predefined movement; processing the first real-time video of the patient to determine a set of health parameters; analyzing the set of health parameters to determine a current fitness state of the patient. The method further includes identifying a set of exercises to be performed by the patient; capturing a second real-time video of the patient performing an exercise; extracting a second AI model to determine a deviation of the patient from a plurality of expected movements associated with the exercise; processing the second real-time video of the patient to determine a set of patient mobility parameters; comparing the set of patient mobility parameters with a set of target mobility parameters; generating feedback for the patient; and rendering the feedback on a rendering device.

### CROSS-REFERENCE TO RELATED APPLICATION

This application claims priority benefits under 35 U.S.C. § 119 (e) to U.S. Non-Provisional application Ser. No. 17/467,374, U.S. Non-Provisional application Ser. No. 17/467,381, and U.S. Non-Provisional application Ser. No. 17/467,386 filed on Sep. 6, 2021, which is hereby incorporated by reference in its entirety.

### TECHNICAL FIELD

This disclosure relates generally to physical fitness, and more particularly to a method and a system for providing remote physiotherapy sessions to patients.

### BACKGROUND

Today, a right work-life balance has become a challenge for people, in this era of rapid urbanization and a fast-paced life. While trying hard to maintain the work-life balance, people often ignore their physical well-being and face difficulty in dedicating a regular time daily in performing physical activities (e.g., exercises). The sedentary nature of many modern jobs and lifestyles has led to a decrease in physical activity levels among people. This lack of physical activity has significant impact on both physical and mental health of a person. People who are less physically active have a higher chance of attracting diseases that may require physiotherapy sessions. People may seek physiotherapy in various situations and for a wide range of conditions, such as rehabilitation after any injury or surgery, chronic pain, musculoskeletal conditions, sports injuries, and the like.

Physiotherapy can be defined as a treatment that the person requires to restore, maintain, and improve his mobility, function, and overall well-being. In particular, physiotherapy helps a person to restore movement and function of a body part that is affected by injury, illness, or disability. Physiotherapy assists individuals suffering from movement impairments that may be congenital (existing at birth), age-related, accidental, or the result of specific lifestyle changes. The field of physiotherapy has evolved significantly, particularly in recent years, with the integration of technology and innovative approaches. Examples of currently existing approached includes, in-person sessions, telehealth and virtual sessions, home exercise programs (HEP), online education and self-management resources, etc. The currently used approach provides several benefits, including increased accessibility, convenience, and reduced barriers to receiving treatment.

However, these current approaches has some challenges, such as, these approaches are inefficient in encouraging patients to take active participation. In addition, these currently used approaches require continuous involvement of a physiotherapist to assist the patient. Moreover, the currently used approaches has made the life of the patient easier, but not of the physiotherapist, as none of the existing approach focuses on providing any assistance to physiotherapist for providing physiotherapy sessions to the patient.

### SUMMARY

In one embodiment, a method for providing remote physiotherapy sessions is disclosed. In one example, the method may include capturing, by at least one camera, a first real-time video of a patient performing at least one predefined movement. The method may further include processing in real-time, by a first Artificial Intelligence (AI) model, the first real-time video of the patient to determine a set of health parameters based on the at least one predefined movement performed by the patient. The method may further include analyzing, by the first AI model, the set of health parameters and at least one of patient health records and demographic data to determine a current fitness state of the patient. The method may further include identifying, by the first AI model, a set of exercises to be performed by the patient, based on the current fitness state of the patient. The method may further include capturing, by the at least one camera, a second real-time video of the patient performing an exercise from the set of exercises. It should be noted that the second real-time video may include a stream of poses and movements made by the patient to perform the exercise. The method may further include extracting a second AI model based on the current fitness state of the patient and the exercise being performed by the patient. It should be noted that, the second AI model may be configured to determine a deviation of the patient from a plurality of expected movements associated with the exercise based on target exercise performance of a healthy specimen. The method may further include processing in real-time, by the second AI model, the second real-time video of the patient to determine a set of patient mobility parameters based on current exercise performance of the patient. The method may further include comparing, by the second AI model, the set of patient mobility parameters with a set of target mobility parameters. It should be noted that the set of target mobility parameters may correspond to the healthy specimen. The method may further include generating, by the second AI model, feedback for the patient based on comparison of the set of patient mobility parameters with the set of target mobility parameters. It should be noted that the feedback may include at least one of corrective actions or alerts. Further, the feedback may be at least one of visual feedback, aural feedback, or haptic feedback. The method may further include rendering, by the second AI model, the feedback on a rendering device.

In another embodiment, a system for providing remote physiotherapy sessions is disclosed. The system may include a processor, and a memory communicatively coupled to the processor. The memory includes processor executable instructions, which when executed by the processor causes the processor to capture, by at least one camera, a first real-time video of a patient performing at least one predefined movement. The processor-executable instructions, on execution, may further cause the processor to process in real-time, by a first Artificial Intelligence (AI) model, the first real-time video of the patient to determine a set of health parameters based on the at least one predefined movement performed by the patient. The processor-executable instructions, on execution, may further cause the processor to analyze, by the first AI model, the set of health parameters and at least one of patient health records and demographic data to determine a current fitness state of the patient. The processor-executable instructions, on execution, may further cause the processor to identify, by the first AI model, a set of exercises to be performed by the patient, based on the current fitness state of the patient. The processor-executable instructions, on execution, may further cause the processor to capture, by the at least one camera, a second real-time video of the patient performing an exercise from the set of exercises. It should be noted that the second real-time video may include a stream of poses and movements made by the patient to perform the exercise. The processor-executable instructions, on execution, may further cause the processor to extract a second AI model based on the current fitness state of the patient and the exercise being performed by the patient. It should be noted that, the second AI model may be configured to determine a deviation of the patient from a plurality of expected movements associated with the exercise based on target exercise performance of a healthy specimen. The processor-executable instructions, on execution, may further cause the processor to process in real-time, by the second AI model, the second real-time video of the patient to determine a set of patient mobility parameters based on current exercise performance of the patient. The processor-executable instructions, on execution, may further cause the processor to compare, by the second AI model, the set of patient mobility parameters with a set of target mobility parameters. It should be noted that, the set of target mobility parameters may correspond to the healthy specimen. The processor-executable instructions, on execution, may further cause the processor to generate, by the second AI model, feedback for the patient based on comparison of the set of patient mobility parameters with the set of target mobility parameters. It should be noted that the feedback may include at least one of corrective actions or alerts. Further, the feedback may be at least one of visual feedback, aural feedback, or haptic feedback. The processor-executable instructions, on execution, may further cause the processor to render, by the second AI model, the feedback on a rendering device.

In yet another embodiment, a non-transitory computer-readable medium storing computer-executable instruction for providing remote physiotherapy sessions is disclosed. The stored instructions, when executed by a processor, may cause the processor to perform operations including capturing a first real-time video of a patient performing at least one predefined movement. The operations may further include processing in real-time, the first real-time video of the patient to determine a set of health parameters based on the at least one predefined movement performed by the patient. The operations may further include analyzing the set of health parameters and at least one of patient health records and demographic data to determine a current fitness state of the patient. The operations may further include identifying a set of exercises to be performed by the patient, based on the current fitness state of the patient. The operations may further include capturing a second real-time video of the patient performing an exercise from the set of exercises. It should be noted that the second real-time video may include a stream of poses and movements made by the patient to perform the exercise. The operations may further include extracting a second AI model based on the current fitness state of the patient and the exercise being performed by the patient. It should be noted that the second AI model may be configured to determine a deviation of the patient from a plurality of expected movements associated with the exercise based on target exercise performance of a healthy specimen. The operations may further include processing in real-time, the second real-time video of the patient to determine a set of patient mobility parameters based on current exercise performance of the patient. The operations may further include comparing the set of patient mobility parameters with a set of target mobility parameters. It should be noted that the set of target mobility parameters may correspond to the healthy specimen. The operations may further include generating feedback for the patient based on comparison of the set of patient mobility parameters with the set of target mobility parameters. It should be noted that the feedback may include at least one of corrective actions or alerts. Further, the feedback may be at least one of visual feedback, aural feedback, or haptic feedback. The operations may further include rendering the feedback on a rendering device.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a block diagram of a system configured for providing remote physiotherapy sessions, in accordance with some embodiment.

FIG. 2 illustrates a flowchart of a method for providing remote physiotherapy sessions, in accordance with some embodiment.

FIG. 3 illustrates a flowchart of a method for receiving user selection of an exercise from a set of exercises, in accordance with some embodiment.

FIG. 4 illustrates a flowchart of a method of rendering feedback to a patient, in accordance with some embodiment.

FIG. 5 illustrates a flowchart of a method of customizing an exercise for a patient, in accordance with some embodiment.

FIG. 6 illustrates a flowchart of a method for suggesting an alternative exercise instead of an exercise to a patient, in accordance with some embodiment.

FIG. 7 illustrates a flowchart of a method of rendering a summarized report to an end user, in accordance with some embodiment.

FIG. 8 illustrates a flowchart of a method for providing an authorization to a patient, in accordance with some embodiment.

FIGS. 9A-9E depicts an exemplary technique of rendering of a set of exercises to a patient, in accordance with some embodiment.

FIGS. 10A and 10B represent an exemplary scenario depicting a technique of capturing real-time videos of a patient, in accordance with an exemplary embodiment.

FIG. 11 represents a GUI displaying current exercise performance and pose skeletal model of a patient, in accordance with an exemplary embodiment.

FIGS. 12A and 12B represent GUIs depicting exercise reports generated based on assigned exercises performed by a patient, in accordance with an exemplary embodiment.

FIG. 13 represents a GUI depicting notifications received by a patient based on assigned exercises, in accordance with an exemplary embodiment.

FIG. 14 represents a GUI depicting a summarized report generated based on monitoring a patient is represented, in accordance with an exemplary embodiment.

FIGS. 15A-15K, depicts an exemplary technique of assisting a physiotherapist for providing remote physiotherapy sessions to a patient, in accordance with some embodiment.

FIG. 16 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

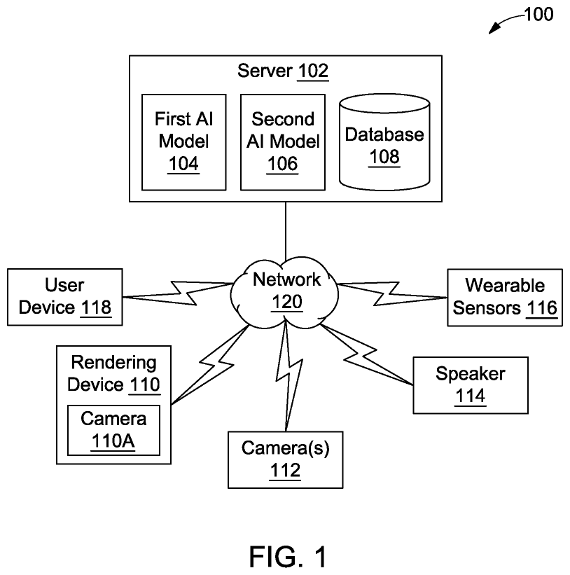

Referring now to FIG. 1, a block diagram of a system 100 configured for providing remote physiotherapy sessions is illustrated, in accordance with some embodiment. In order to provide remote physiotherapy sessions to patients, the system 100 may include a server 102. The server 102 may be configured to provide remote physiotherapy sessions to the patients. In order to provide the remote physiotherapy sessions to a patient, the server 102 may include a memory and a processor. The memory may further include a first Artificial Intelligence (AI) model 104, a second AI model 106, and a database 108. Further, the memory may store instructions that, when executed by the one or more processors, cause the one or more processors to provide remote physiotherapy sessions to the patient, in accordance with aspects of the present disclosure.

By way of an example, suppose the patient may be suffering from shoulder pain for which he might be looking for a remote treatment via the remote physiotherapy sessions. In this case, to provide the remote physiotherapy sessions to the patient, the patient may interact with the server 102 using his communication device, i.e., a rendering device 110 over a network 120. In some embodiment, the patient may interact with the server 102 via his smartphone (wired or wirelessly connected to the rendering device 110) over the network 120. In some embodiment, the rendering device 110 may be configured in such a way that it may provide the remote physiotherapy sessions to the patient without requirement of connection with the server 102. In other words, the rendering device 110 may have intelligence of providing the remote physiotherapy to the patient.

The network 120, for example, may be any wired or wireless communication network and the examples may include, but are not limited to, the Internet, Wireless Local Area Network (WLAN), Wi-Fi, Long Term Evolution (LTE), Worldwide Interoperability for Microwave Access (WiMAX), and General Packet Radio Service (GPRS). Further, examples of the rendering device 110 may include, but is not limited to, a smart TV, an Augmented Reality (AR) device, a Virtual Reality (VR) device, a mobile phone, a laptop, a tablet, a smart mirror, a smart projector with inbuilt camera, or any computing device.

In order to interact with the server 102, initially, the patient may login or signup in an application installed on the rendering device 110 using his associated credentials, that is hosted on the server 102. Upon login, the patient may select ‘a shoulder pain symptom’ from a list of symptoms rendered to the patient via the rendering device 110. In other words, when the patient might have been feeling some discomfort in his shoulder for some time, then the patient may login/signup in the application and select the shoulder pain symptom to receive assistance for his should pain. In some another embodiment, the patient might have taken consultation from a physiotherapist either remotely (i.e., by using the installed application) or by physically visiting the physiotherapist. Further, based on initial diagnosis done by the physiotherapist, the patient might be aware about his reason of discomfort and may accordingly select one or more symptoms from the list of symptoms. It should be noted that the list of physiotherapy symptoms may be stored within the database 108 of the server 102. In an embodiment, the patient selection may include a gesture, a touch, or an audio command.

Further, upon selecting ‘the shoulder pain symptom’, the server 102 may provide an instruction to perform at least one predefined movement. The provided instruction may be rendered to the patient via the rendering device 110. Further, while the patient might be performing the at least one pre-defined movement, a camera 110A of the rendering device 110 may be configured to capture a first real-time video of the patient. The rendering device 110 may be configured to send the first real-time video to the server 102 via the network 120. In some embodiment, the first real time video may be captured via at least one camera 112 communicatively coupled to the rendering device 110 and the server 102.

Upon receiving the first real-time video, the first AI model 104 may be configured to process the first real-time video of the patient. The processing of the first real-time video may be done to determine a set of health parameters. The set of health parameters of the patient may be determined based on the at least one predefined movement performed by the patient. Examples of the set of health parameters may include blood pressure, body temperature, pulse rate, heart rate, oxygen saturation, or breathing rate of the patient while performing the at least one pre-defined movement, and movement or range of motion of body part requiring treatment, muscular strength, and the like. In some embodiment, the set of health parameters may be captured using a set of wearable sensors 116. Examples of the set of wearable sensors 116 may include, but is not limited to, an Electrocardiogram (ECG) sensor, an Electroencephalogram (EEG) sensor, an Electromyography (EMG) sensor, a pulse oximeter, and the like.

In continuation to the above example, when the patient requires treatment for the shoulder discomfort, in the case, the patient may be instructed to perform the at least one predefined movement to determine the set of health parameters in order to diagnose the shoulder discomfort of the patient. In other words, the patient may be asked to perform the at least one predefined movement to determine the blood pressure, the heart rate, and a level of the movement of a corresponding arm with the shoulder pain, or the range of motion of the corresponding arm, and the like. For example, the at least one predefined movement that the patient might have been instructed to do may be to “move arm back and forth of the corresponding shoulder with pain”. By way another example, the at least one predefined movement instructed to the patient may be to “move the arm of the corresponding shoulder in a circulation motion”.

Once the set of health parameters is determined, the first AI model 104 may be configured to analyze each of the set of health parameters. In addition to the set of health parameters, the first AI model 104 may be configured to analyze at least one of patient health records and demographic data to determine a current fitness state of the patient. Examples of the patient health records may include, but is not limited to, known allergic reactions including drug allergies, chronic disease, family medical history, imaging reports (e.g., X-rays), medications and dosing, prescription record, surgeries and other procedures, list and dates of illness and hospitalizations, and the like. Examples of demographic data of the patient may include, but are not limited to, name, age, gender, email address, date of birth, phone number, insurance information (e.g., insurance number), and the like.

Further, based on the analysis of the set of health parameters along with the patient health record and demographic data, the first AI model 104 may be configured to determine the current fitness state of the patient. Once the current fitness state is determined, the first AI model 104 may be configured to identify the set of exercises to be performed by the patient, based on the current fitness state of the patient. In continuation to the above example, suppose based on the analysis, the current fitness state of the patient is determined to be ‘bursitis shoulder’. In this case, based on the analysis, the set of exercises determined by the first AI model 104 that needs to be performed by the patient for ‘bursitis shoulder’ condition may be ‘overhead stretch’, ‘shoulder blade’, and ‘cross arm stretch’.

Once the set of exercises is identified by the first AI model 104, the server 102 may be configured to send the identified set of exercises to the rendering device 110 through the network 120. Further, the rendering device 110 may be configured to render the set of exercises assigned to the patient via a Graphical User Interface (GUI) of the rendering device 110. The patient may then select an exercise (for example: overhead stretch) from the set of exercises that the patient may perform first. In an embodiment, upon selecting the exercise, the patient may be able to see an instructional video based on his requirement. It should be noted that the patient selection may include a gesture, a touch, or an audio command. Further, based on the patient selection of the exercise ‘an instructional video option’ may be available corresponding to the exercise. As will be appreciated, the instructional video corresponding to a plurality of exercises associated with a plurality of physiotherapy treatments may be stored with the database 108 of the server 102.

After seeing the instructional video, once the patient starts performing the exercise, then the camera 110A of the rendering device 110 or the at least one camera 112 may be configured to capture a second real-time video of the patient. The second real-time video may be captured while the patient may be performing the exercise. In an embodiment, the second real-time video may include a stream of poses and movements made by the patient to perform the exercise. As will be appreciated, in some embodiment, the at least one camera 112 may be used for facial recognition of the patient. Facial data corresponding to the patient is associated with the patient's profile. The patient profile is stored in the database 108 and may be associated with current and historical patient data such as, but not limited to, history of physiotherapy treatment, custom settings, messages, profile data, etc. In an embodiment, the patient profile may be secured using any biometric authentication methods.

Once the second real-time video is captured, the server 102 may be configured to extract the second AI model 106. The second AI model 106 may be extracted based on the current fitness state of the patient and the exercise being performed by the patient. The second AI model 106 extracted by the server 102 may be configured to determine a deviation of the patient from a plurality of expected movements associated with the exercise based on target exercise performance of a healthy specimen. As will be appreciated, an expected movement of the target exercise performed by the healthy specimen may correspond to a correct way in which the exercise is performed by the healthy specimen (e.g., an exercise expert). In an embodiment, the target exercise performance may be a video recording of the exercise expert, a 3-Dimensional (3D) model of the exercise expert, a 2-Dimensional (2D) model, or a 4-Dimensional (4D) of the exercise expert.

In an embodiment, the determined deviation may be used by the second AI model 106 to compute a degree of movement. The degree of movement may be computed for each identified exercise for each session. As will be appreciated, the degree of movement may provide information with respect to improvement in the condition of the patient during each session. In continuation to the above example, suppose for the bursitis shoulder condition determined for the patient, the set of three exercises, i.e., ‘overhead stretch’, ‘shoulder blade’, and ‘cross arm stretch’ is identified for the patient. Further, each of the set of three exercises is customized, such that, the patient needs to perform each of the set of three exercises ‘5 times’ with ‘3 sets’ in a day in the beginner mode for a week. In this scenario, each day of the week in which the patient may perform each of the set of three exercises ‘5 times’ with ‘3 sets’ may correspond to a session. In this case, during a 1^{st} session, while the patient may be performing the overhead stretch exercise, the patient only be able to raise his arms upwards to a few degrees (e.g., 40 degrees). However, till 5^{th} session, the patient may be able to raise his arm upwards to 80 degrees. Then, in this case, based on the improvement done by the patient, the degree of movement, i.e., 80 degrees for the overhead stretch exercise may be rendered to the patient. In some embodiment, the degree of movement, i.e., 80 degrees may be rendered to the physiotherapist for evaluating progress in the patient's condition with respect to bursitis shoulder condition.

Once the second AI model 106 is extracted, the second AI model 106 may be configured to process the second real-time video in real-time. In other words, the second AI model 106 may be configured to process the second real-time video that is being captured by the camera 110A or the at least one camera 112 while the patient may be performing the exercise. In an embodiment, the second AI model 106 may process the second real-time video to determine a set of patient mobility parameters based on current exercise performance of the patient. Examples of the set of patient mobility parameters may include, but are not limited to, flexibility, balance, coordination, range of motion, time, speed, posture, form of the exercise, and the like. It should be noted that, the rendering device 110 may include one or more in-built sensors (for example, proximity sensor, audio sensor, Light Detection and Ranging (LIDAR) sensor, Infrared (IR) sensor, and other motion-based sensors) to receive additional data that may be processed and analyzed for the patient.

Further, the second AI model 106 may be configured to compare the set of patient mobility parameters with a set of target mobility parameters. The set of target mobility parameters may be accurate mobility parameters, for example, correct form of performing the exercise. In an embodiment, the set of target mobility parameters may be of the healthy specimen. In order to perform the comparison, the second AI model 106 may overlay the patient in the second real-time video with a pose skeletal model. The pose skeletal model may include a plurality of key points based on the exercise. Each of the plurality of key points may be overlayed over a corresponding joint of the patient in the second real-time video.

Further, based on the comparison of the set of patient mobility parameters with the set of target mobility parameters, the second AI model 106 may be configured to generate feedback for the patient. The feedback may include at least one of corrective actions or alerts. The feedback may be at least one of visual feedback, aural feedback, or haptic feedback. In an embodiment, the feedback may include generation of a warning to patient. The warning may be an indication for correcting the current pose of the patient, and an indication for correcting motion associated with the current pose of the patient.

Once the feedback is generated, the second AI model 106 may render the feedback on the rendering device 110. In particular, the second AI model 106 may render the feedback on the GUI of the rendering device 110 to the patient. Rendering the feedback includes overlaying one of at least one corrective action over the pose skeletal model overlayed on the second real-time video of the user. Rendering the feedback further includes displaying the alerts on the GUI of the rendering device. Rendering the feedback further includes outputting the aural feedback to the user, via a speaker.

In some embodiment, the feedback may be generated and rendered based on the degree of movement computed for each exercise performed by the patient. In particular, modulation of the feedback may vary based on the improvement determined using the computed degree of movement. For example, the modulation of the feedback may be high, when the degree of movement is high, and the modulation of the feedback may be low, when the degree of movement is low. In other words, the better the degree of movement, the higher is the modulation of the feedback. In continuation to the above example, for the bursitis shoulder condition determined for the patient, during the first session, when the patient was able to raise his arms upwards to 40 degrees while performing the overhead stretch exercise. In this case, when the feedback is the aural feedback, then the pitch (or volume) used to output the aural feedback may be comparatively lower as the computed degree of movement (i.e., 40 degrees) is lower than an accurate degree of movement (100 degrees). However, till 5^{th} session, when the patient is able to raise his arm upwards to 80 degrees (i.e., the computed degree of movement). Then, in this case, based on the improvement done by the patient, the pitch (or the volume) may be comparatively higher than the pitch used to output the aural feedback of 40 degrees. In other words, the volume used to output the aural feedback may be automatically adjusted based on the degree of movement computed for each exercise performed by the patient.

In some embodiment, based on the comparison of the set of patient mobility parameters with the set of target mobility parameters, the second AI model 106 may be configured to customize the exercise for the patient. As will be appreciated, this may be done to ensure that the patient may be able to achieve the set of target mobility parameters. In order to customize the exercise for the patient, a number of repetitions and a number of sets of the exercise may be defined for the patient. In addition, one of a plurality of modes may be selected for the exercise. A mode from the plurality of modes may be selected based on the current fitness state of the patient. In some another embodiment, once the set of exercises is identified, the second AI model 106 may be configured to customize each of the set of exercises.

In addition to rendering the feedback, the second AI model 106 may be configured to identify a failure in completion of the exercise by the patient. In order to identify the failure, the second AI model 106 may be configured to monitor each of the set of exercises being performed by the patient based on a corresponding second real-time video of the patient. In continuation to the above example, suppose the patient is assigned the set of three exercises, i.e., ‘overhead stretch’, ‘shoulder blade’, and ‘cross arm stretch’ for the bursitis shoulder condition, and each of the set of three exercises is customized, such that, the patient needs to perform each of the set of three exercises ‘5 times’ with ‘3 sets’ in a day in the beginner mode for a week.

In this case, based on the monitoring, when the second AI model 106 is not able to obtain the second real-time video of at least one of the set of exercises that needs to be captured by the camera 110A or the at least camera 112. Then in this case, the failure in performing the at least one of the set of exercises is determined by the second AI model 106. In another case, based on the monitoring, when the second AI model 106 is not able to obtain the second real-time video of each of the set of exercises for a day that needs to be captured by the camera 110A or the at least camera 112. Then in this case, the failure in performing each of the set of exercises for that day is determined by the second AI model 106.

Upon identifying the failure, the second AI model 106 may send a reminder to the patient after expiry of a pre-defined time interval for completing of the at least one of the set of exercises. In another case, upon identifying the failure, the second AI model 106 may send a reminder to the patient after expiry of a pre-defined time interval (for example: two consecutive days without exercises) for completing of the set of exercises. Further, based on the monitoring, the second AI model 106 may be configured to generate a summarized report for each of the set of exercises performed by the patient. Further, the summarized report generated by the second AI model 106 may be rendered to the patient via the GUI of the rendering device 110.

Further, based on the summarized report, the second AI model 106 may be able to validate performance of the patient. Furthermore, based on the validation, the second AI model 106 may be configured to provide an authorization to the patient to perform one or more actions. In continuation to above example, when the patient with the bursitis shoulder condition, is able to complete all physiotherapy sessions of each of the set of three exercises successfully, then the patient may be validated to claim an insurance for the treatment provided for the bursitis shoulder condition that caused the shoulder pain.

In some embodiment, in addition to the patient, the generated summarized report may be rendered to an end user, i.e., a physiotherapist via a user device 118. The physiotherapist may be able to analyze the summarized report of the patient. The physiotherapist may analyze the summarized report to evaluate fitness state of the patient after performing the required physiotherapy sessions or validate the patient performance based on the summarized report generated by the second AI model 106. As will be appreciated, the server 102 may assist the physiotherapist in providing treatment to the patient based on the current fitness state determined for the patient. By way of an example, in some embodiment, in order to identify the set of exercises that needs to be performed by the patient based on the determined current fitness state, the first AI model 104 may determine a plurality of exercises for the patient. The plurality of exercises may be rendered by the server 102 to the physiotherapist via the user device 118. Further, the physiotherapist may select the set of exercises that need to be performed by the patient based on the current fitness state of the patient determined by the first AI model 104.

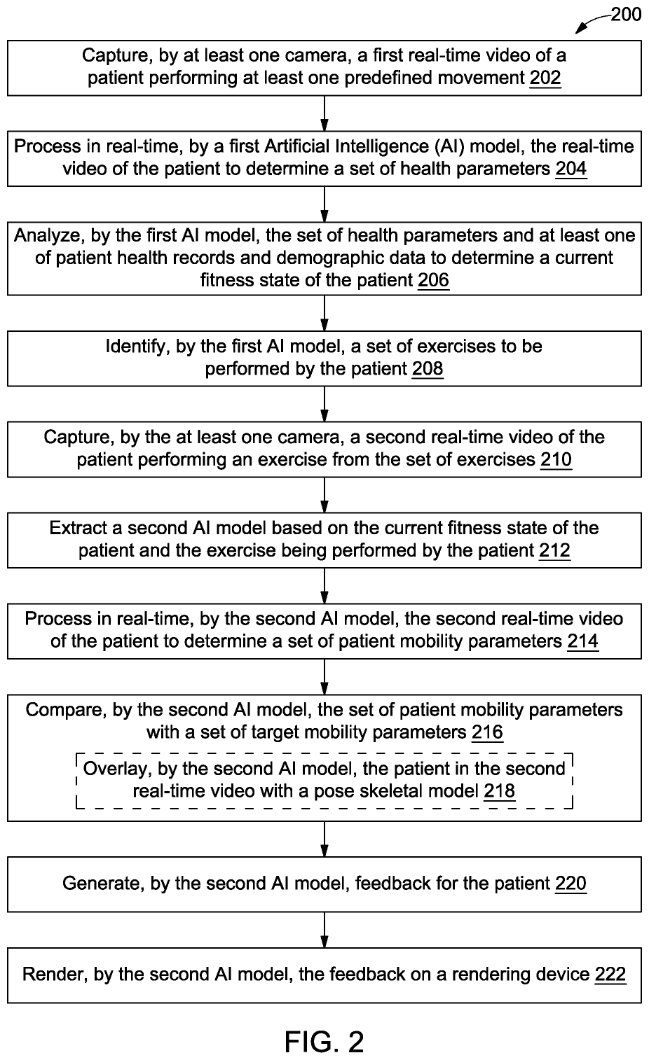

In some another embodiment, the server 102 may assist the physiotherapist in customizing each of the set of exercises for the patient based on comparison of the set of patient mobility parameters with the set of target mobility parameters done by the second AI model 106. By way of an example, the second AI model 106 may suggest the number of repetitions and the number of sets for each of the set of exercises to the physiotherapist. Further, based on the suggestions and the determined current fitness state, the physiotherapist may select the number of repetitions and the number of sets for each of the set of exercises for the patient. By way of an example, the second AI model 106 may suggest one of the plurality of modes to the physiotherapist for each exercise based on the current fitness state of the patient. Further, based on the suggestion and the set of health parameters, the physiotherapist may select a suitable mode (for example: a beginner mode) for the patient. This complete method of providing remote physiotherapy sessions to the patient is further explained in detail in conjunction with FIGS. 2-15K.

Referring now to FIG. 2, a flowchart of a method 200 for providing remote physiotherapy sessions is illustrated, in accordance with some embodiment. FIG. 2 is explained in conjunction with FIG. 1.

In order to provide remote physiotherapy sessions to a patient, at step 202, a first real-time video of the patient may be captured. The first real-time video may be captured while performing at least one predefined movement. In an embodiment, the first real time video may be captured via at least one camera. With reference to FIG. 2, the at least one camera may correspond to the camera 110A of the rendering device 110 or the at least one camera 112.

Upon capturing the at least one video, at step 204, the first real-time video of the patient may be processed in real-time. In an embodiment, the first real-time video may be processed to determine a set of health parameters. The set of health parameters may be determined based on the at least one predefined movement performed by the patient. Examples of the set of health parameters may include blood pressure, body temperature, pulse rate, or breathing rate of the patient while performing the at least one pre-defined movement, and movement or range of motion of body part requiring treatment, muscular strength. With reference to FIG. 2, the set of health parameters may be determined by the first AI model 104.

Further, based on the processing, at step 206, the set of health parameters and at least one of patient health records and demographic data may be analyzed. The set of health parameters and at least one of patient health records and demographic data may be analyzed may be analyzed to determine a current fitness state of the patient. By way of an example, the patient health records may include, but is not limited to, known allergic reactions including drug allergies, chronic disease, family medical history, imaging reports (e.g., X-rays), medications and dosing, prescription record, surgeries and other procedures, list and dates of illness and hospitalizations, and the like. Further, examples of the demographic data of the patient may include, but are not limited to, name, age, gender, email address, date of birth, phone number, insurance information (e.g., insurance number), and the like. With reference to FIG. 2, the current fitness state of the patient may be determined by the first AI model 104.

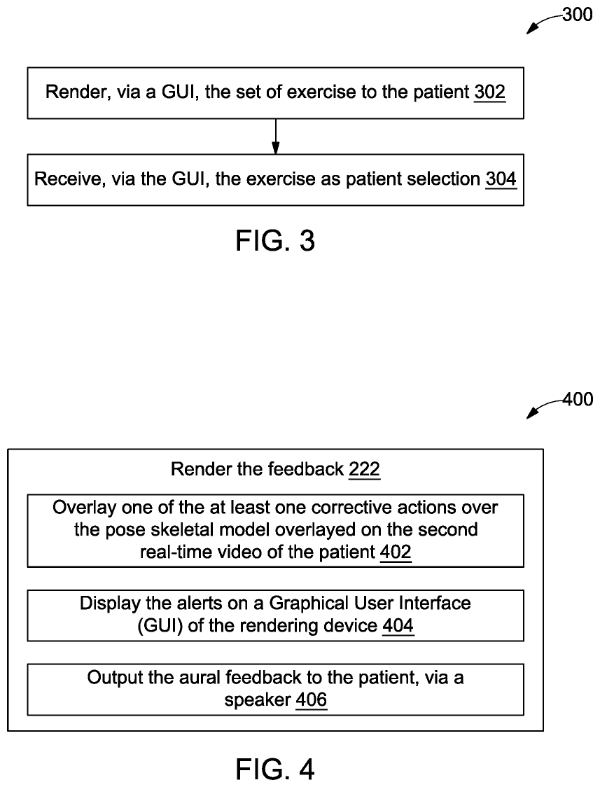

Upon determining the current fitness state of the patient, at step 208, a set of exercises to be performed by the patient may be determined. With reference to FIG. 2, the set of exercises may be determined by the first AI model 104 as per the current fitness state of the patient. Once the set of exercises is determined, each of the set of exercises may be rendered to the user. This is further explained in detail in conjunction with FIG. 3. Further, based on the rendering, once the patient starts performing an exercise from the set of exercises, then at step 210, a second real-time video of the patient may be captured. The second real-time video may be captured while the patient may be performing the exercise. Further, the second the real-time video may include a stream of poses and movements made by the patient to perform the exercise. With reference to FIG. 2, the second real-time video may be captured via the at least one camera. With reference to FIG. 2, the at least one camera may correspond to the camera 110A or the at least one camera 112.

Upon capturing the second real-time video, at step 212, a second AI model may be extracted. The second AI model may be extracted based on the current fitness state of the patient and the exercise being performed by the patient. In an embodiment, the second AI model may be configured to determine a deviation of the patient from a plurality of expected movements associated with the exercise based on target exercise performance of a healthy specimen. In an embodiment, the determined deviation may be used to compute a degree of movement. The degree of movement may be computed for each identified exercise for each session. As will be appreciated, the degree of movement may provide information with respect to improvement in the condition of the patient during each session. As will be appreciated, an expected movement of the target exercise performed by the healthy specimen may correspond to an accurate way in which the exercise is performed by the healthy specimen (e.g., an exercise expert). With reference to FIG. 1, the second AI model may correspond to the second AI model 106.

In order to determine the deviation, at step 214, the second real-time video of the patient may be processed. The second real-time video may be processed by the second AI model. Further, based on processing, a set of patient mobility parameters may be determined based on current exercise performance of the patient. By way of an example, the set of patient mobility parameters may include, but are not limited to, flexibility, balance, coordination, range of motion, time, speed, posture, form of the exercise, and the like. Further, at step 216, the set of patient mobility parameters may be compared with a set of target mobility parameters. The set of target mobility parameters may correspond to the healthy specimen. In other words, the set of target mobility parameters may be accurate mobility parameters, for example, correct form of performing the exercise.

In order to perform the comparison, at step 218, the patient in the second real-time video may be overlayed with a pose skeletal model. The pose skeletal model may include a plurality of key points based on the exercise. Each of the plurality of key points may be overlayed over a corresponding joint of the patient in the second real-time video. Further based on the comparison of the set of patient mobility parameters with the set of target mobility parameters, at step 220, feedback for the patient may be generated. The feedback may include at least one of corrective actions or alerts. Further, the feedback may include at least one of visual feedback, aural feedback, or haptic feedback. In some embodiment, the feedback may include generation of a warning to the patient. The warning may be an indication for correcting the current pose of the patient, and an indication for correcting motion associated with the current pose of the patient. Once the feedback is generated, at step 222, the generated feedback may be rendered on a rendering device. In particular, the generated feedback may be rendered to the patient via his rendering device, i.e., the rendering device 110.

As will be appreciated, in some embodiment, the feedback may be generated and rendered based on the degree of movement computed for each exercise performed by the patient. In particular, modulation of the feedback (i.e., visual feedback, aural feedback, or haptic feedback) may vary based on the improvement determined using the computed degree of movement. For example, the modulation of the feedback may be high, when the degree of movement is high, and the modulation of the feedback may be low, when the degree of movement is low. In other words, the better the degree of movement, the higher is the modulation of the feedback. In other words, in case of the aural feedback, the volume (or the pitch) used to output the aural feedback may be automatically adjusted based on the degree of movement computed for each exercise performed by the patient. Similarly, in the case of the haptic feedback, an intensity of vibration may be automatically adjusted based on the degree of movement computed for each exercise performed by the patient.

Referring now to FIG. 3, a flowchart of a method 300 for receiving user selection of an exercise from a set of exercises is illustrated, in accordance with some embodiment. FIG. 3 is explained in conjunction with FIGS. 1 and 2.

With reference to FIG. 2, as mentioned via the step 208, once the set of exercises is determined, then at step 302, the set of exercises may be rendered to the patient. The set of exercises may be rendered to the patient on the GUI of the rendering device 110. Further, upon rendering each of the set of exercises, at step 304, patient selection of the exercise from the set of exercises may be received via the GUI. In particular, the first AI model 104 may be configured to receive the patient selection of the exercise.

Upon receiving the patient selection of the exercise, the patient may be able to see an instructional video based on his requirement. It should be noted that the patient selection may include a gesture, a touch, or an audio command. Further, based on the patient selection of the exercise ‘an instructional video option’ may be available corresponding to the exercise. The patient may select ‘the instructional video option’ to see the instructional video of the selected exercise. As will be appreciated, the instructional video corresponding to a plurality of exercises associated with each physiotherapy treatment may be stored with a database (same as the database 108).

Referring now to FIG. 4, a flowchart of a method 400 of rendering feedback to a patient is illustrated, in accordance with some embodiment. FIG. 4 is explained in conjunction with FIGS. 1-3.

With reference to FIG. 2, in order to render the feedback to the patient as mentioned via the step 222, at step 402, one of at least one corrective action may be overlayed over the pose skeletal model overlayed on the second real-time video of the patient. In other words, in order to provide the feedback to the patient, while the patient may be performing the exercise, the second real-time video being captured may be processed and compared in the real-time. Further, based on the processing and the comparison, the at least one corrective action may be overlayed over the pose skeleton model that is overlayed on the second real-time video being captured while the patient is performing the exercise.

By way of an example, while the user may be performing the exercise ‘stretch your arms straight in upward direction’, a left arm of the patient may not be straight. Then, in this case, the at least one corrective action may be correct position of the left arm overlayed over the pose skeleton model that is overlayed on the second real-time video of the patient being captured while the patient may be performing the exercise ‘stretch your arms straight in upward direction’. Further, at step 404, the alerts may be displayed to the patient via the GUI of the rendering device 110. In continuation to the above example, the alerts may be, for example, displaying of correct position of the left arm, rendering a notification stating, ‘keep you elbow straight of the left arm’, and the like. Further, at step 406, the aural feedback may be outputted to the patient, via a speaker. The speaker may be communicatively coupled with the rendering device 110 and the server 102. In an embodiment, the feedback may include generating a warning to the patient. The warning may be indicative of correction of the current pose of the patient or indicative of correction of motion associated with a current pose of the patient.

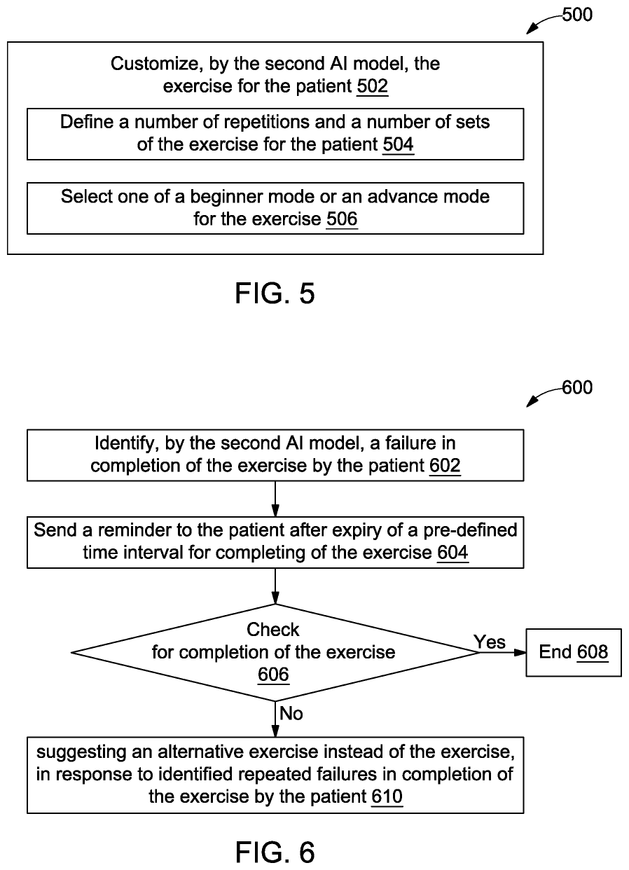

Referring now to FIG. 5, a flowchart of a method 500 of customizing an exercise for a patient is illustrated, in accordance with some embodiment. FIG. 5 is explained in conjunction with FIGS. 1-4.

At step 502, the exercise for the patient may be customized. In an embodiment, the exercise may be customized based on comparison of the set of patient mobility parameters with the set of target mobility parameters. It should be noted that the exercise may be customized by the second AI model 106. In some embodiment, the second AI model 106 may assist a physiotherapist to customize the exercise for the patient based on the comparison. In order to customize the exercise, at step 504, a number of repetitions and a number of sets of the exercise for the patient may be defined.

Further, at step 506, one of a plurality of modes may be selected for the exercise. In an embodiment, a mode from the plurality of modes may be selected based on the current fitness state of the patient. For example, a beginner mode may be selected when the patient may be performing the exercise for a first time (i.e., first session) and is not habitual of performing assigned exercises. Whereas an intermediate mode may be selected when the patient may have performed the exercise for few sessions and a current fitness state (improved fitness state) of the patient might have improved from a previous fitness state of the patient that was determined before the start of the first session. As will be appreciated, the customization may be done for each of the set of exercises.

Referring now to FIG. 6, a flowchart of a method 600 for suggesting an alternative exercise instead of an exercise to a patient is illustrated, in accordance with some embodiment. FIG. 6 is explained in conjunction with FIGS. 1-5.

At step 602, a failure in completion of the exercise by the patient may be identified. With reference to FIG. 1, the failure in the completion of the exercise may be identified by the second AI model 106. Further, upon identifying the failure, at step 604, a reminder may be sent to the patient. In an embodiment, the reminder may be sent after expiry of a pre-defined time interval for completing the exercise, in response to identifying the failure in completion of the exercise by the patient. By way of an example, the pre-defined time interval may be of 1 hour, e.g., 1 hour post daily exercise time, or 1 day, e.g., day when the patient may not have performed the exercise or each of the set of exercises.

Upon sending the reminder, at step 606, a check may be performed to determine completion of the exercise. In other words, a check may be performed to determine whether the patient has performed the exercise after the reminder. In one embodiment, based on the check performed, if the patient might have performed the exercise, then at step 608, the method 300 may end. As will be appreciated, the identification of the failure for the exercise may be performed for each of the set of exercises identified for the patient. In another embodiment, based on the check performed, if the patient may have not performed the exercise even after the reminder, then upon identifying repeated failure in completion of the exercise, at step 610, an alternative exercise may be suggested to the patient instead of the exercise.

By way of an example, with reference to FIG. 1, once the set of exercises is identified and customized, then the second AI model 106 may be configured to monitor completion of each of the set of exercises by the patient. In order to monitor, the second AI model 106 may be configured to capture and process the second real-time video of the patient captured by the camera 110 or the at least one camera 112. Now suppose the patient may not have performed an exercise from a set of three exercises for a day, then, the reminder may be sent to the patient to perform the exercise. However, even after the reminder, the patient might have performed only the first two exercises for the set of three exercises, for 2 consecutive days, then an alternative exercise for third exercise may be suggested to the patient.

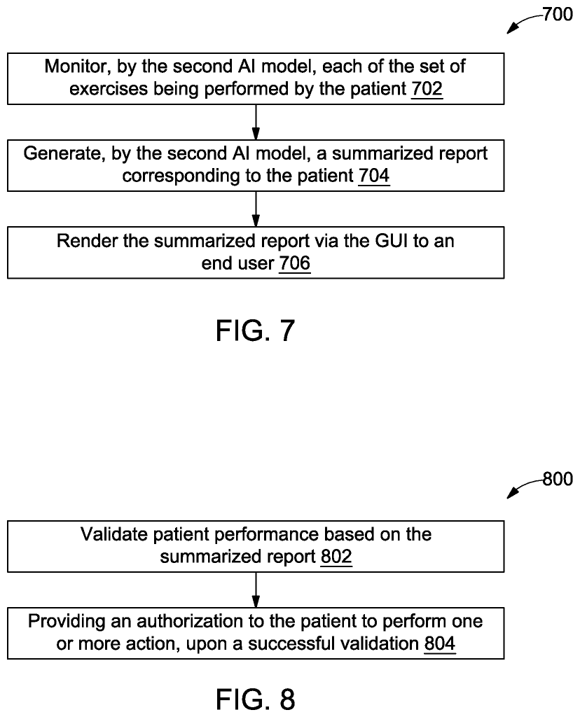

Referring now to FIG. 7, a flowchart of a method 700 of rendering a summarized report to an end user is illustrated, in accordance with some embodiment. FIG. 7 is explained in conjunction with FIGS. 1-6.

Once each of the set of exercises is identified and rendered to the patient, then at step 702, each of the set of exercises being performed by the patient may be monitored. In an embodiment, each of the set of exercises being performed by the patient may be monitored based on a corresponding second real-time video of the patient. In other words, each of the set of exercises may be monitored based on the second real-time video captured by the at least one camera 112 or the camera 110A corresponding to each exercise being performed by the patient. With reference to FIG. 1, the monitoring of each of the set of exercises may be done by the second AI model 106.

Further, based on the monitoring, at step 704, a summarized report corresponding to the patient may be generated. In an embodiment, the summarized report may include progress details (e.g., improvement in patient's condition) and performance details (e.g., accuracy of performing each exercise, duration of performing each exercise, calories burnt, etc.). Further, at step 706, the generated summarized report may be rendered via the GUI to the patient. With reference to FIG. 1, the summarized report may be rendered to the patient via the GUI of the rendering device 110.

The patient may utilize the summarized report to view his progress and performance. It should be noted that the summarized report may be generated based on pre-defined criteria. The pre-defined criteria may be generating the summarized report weekly, i.e., 7 days, generating the summarized report after 15 days, generating the summarized report, once a month, generating the summarized report after every session, and the like. In some embodiment, the generated summarized report may be rendered to the physiotherapist, i.e., the end user, via the GUI of the user device 118. The physiotherapist may utilize the summarized report to monitor progress and performance of the patient. Further, based on the summarized report, the physiotherapist may evaluate a current fitness state (improved condition) of the patient.

Referring now to FIG. 8, a flowchart of a method 800 for providing an authorization to a patient is illustrated, in accordance with some embodiment. FIG. 8 is explained in conjunction with FIGS. 1-7.

With reference to FIG. 7, once the summarized report is generated as mentioned via step 704, then at step 802, patient's performance may be validated based on the summarized report. With reference to FIG. 1, the performance of the patient may be validated by the second AI model 106. In some embodiment, the second AI model 106 may assist the physiotherapist to validate the performance of the patient. Further, based on the validation of the patient's performance, at step 804, an authorization may be provided to the patient to perform one or more actions upon a successful validation.

By way of an example, based on the generated summarized report, the second AI model 106 may identify whether the patient has performed each of the set of exercises that were identified for him. In addition, the second AI model 106 may validate whether the patient has completed all sessions of each of the set of exercises required for completing the treatment. In a first embodiment, when the second AI model 106 identifies completion of all sessions of each of the set of exercises by the patient, then the patient's performance may be marked as the successful validation. In second embodiment, when the second AI model 106 identifies incompletion in at least one sessions or incompletion of at least one exercise of the set of exercises by the patient, then the patient's performance may be marked as an unsuccessful validation.

Further, in the first embodiment, based on the successful validation, the authorization of the one or more actions may be provided to the patients. The one or more actions may be, such as, the patient may be able to claim existing insurance for paying cost of a physiotherapy treatment or purchase an insurance with broader coverage including physiotherapy treatment required for a wide variety of reasons for various body parts. In the second embodiment, based on the unsuccessful validation, the authorization of the one or more actions may not be provided to the patients. For example, the patient may be able to claim existing insurance for paying the cost of the physiotherapy treatment or may have limited future insurance coverage including physiotherapy treatment for fewer body parts. By way of another example, the second AI model 106 may render incompletion of at least one of the set of exercises to the physiotherapist, based on which the physiotherapist may take provide authorization of the one or more actions to the patient.

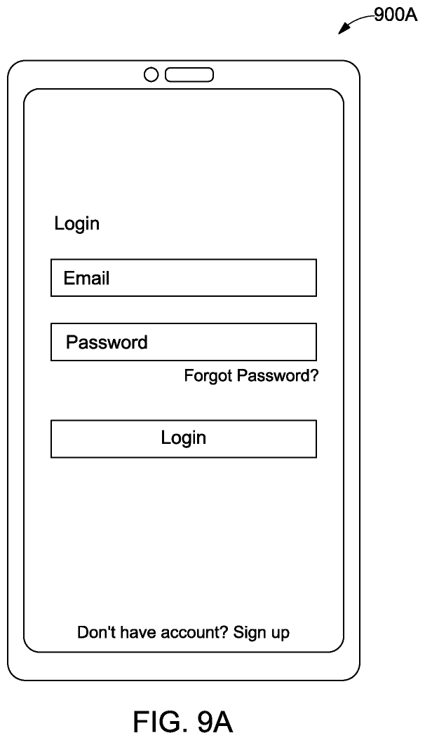

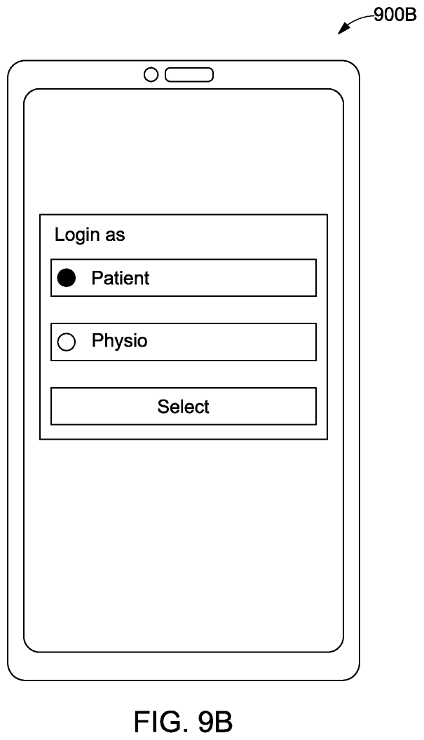

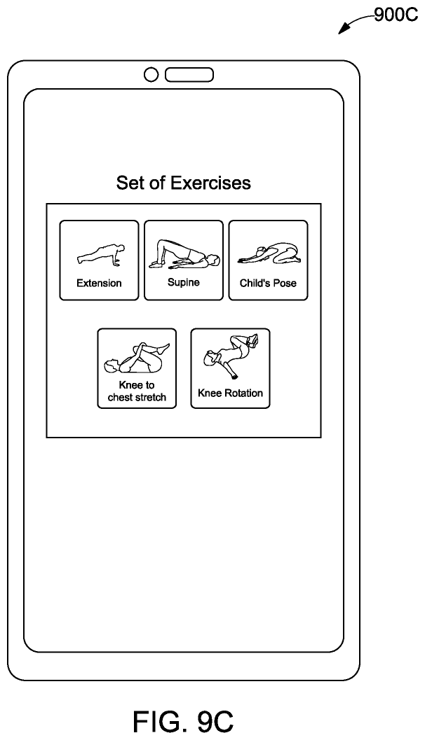

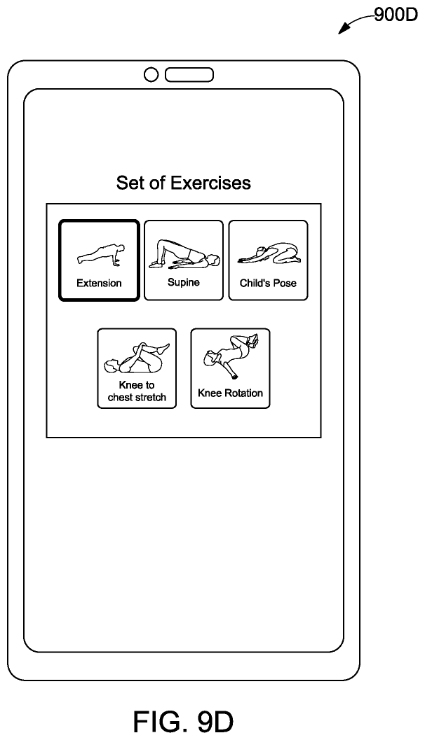

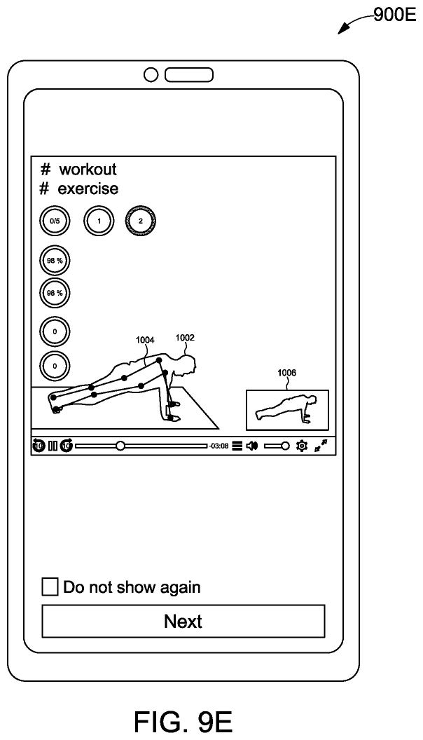

Referring now to FIG. 9A-9E, an exemplary technique of rendering of a set of exercises to a patient is depicted, in accordance with an exemplary embodiment. FIGS. 9A-9D are explained in conjunction with FIGS. 1-8.

With reference to FIG. 1, the GUIs depicted via the FIG. 9A-9E may be the GUIs of the rendering device 110. In some embodiment, the GUIs may be the GUIs of a user device (e.g., a smartphone, a laptop, a tablet, and the like) communicatively coupled to the rendering device 110 (e.g., a smart mirror). By way of an example, consider a scenario when the patient may be interested in taking a remote physiotherapy treatment, i.e., remote physiotherapy sessions for back pain issue, then the patient may need to connect to the server 102. In order to connect to the server 102, initially the patient may download an application on the rendering device 110 or the user device (e.g., his smartphone).

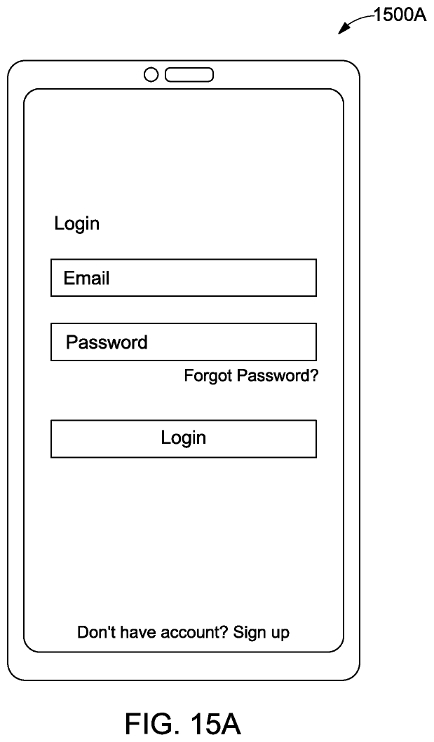

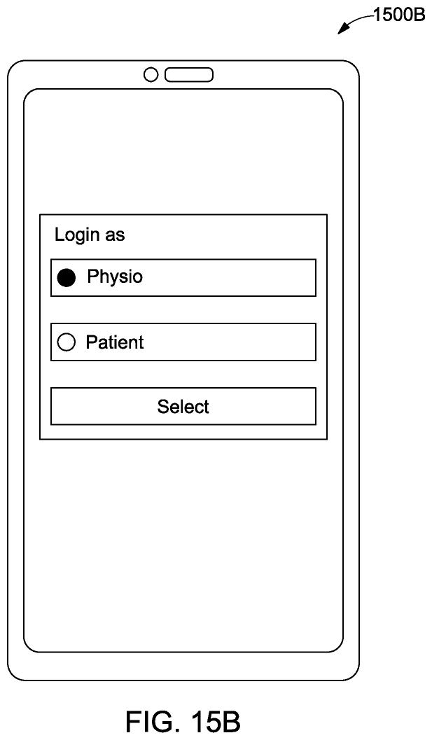

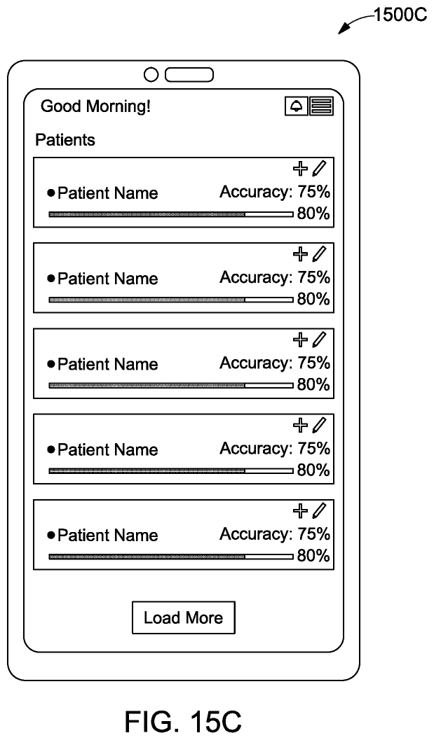

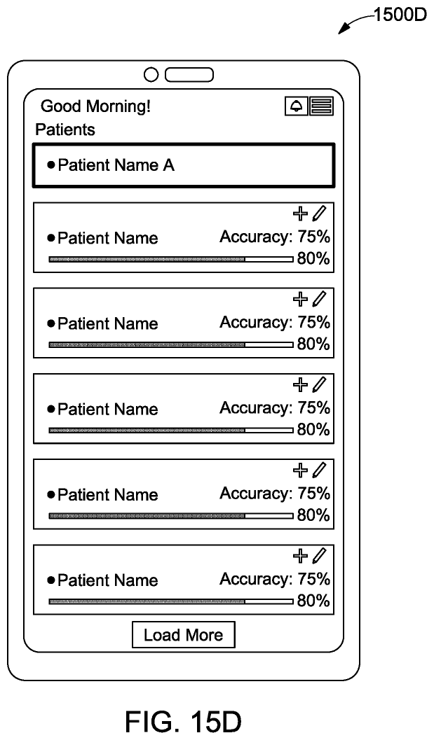

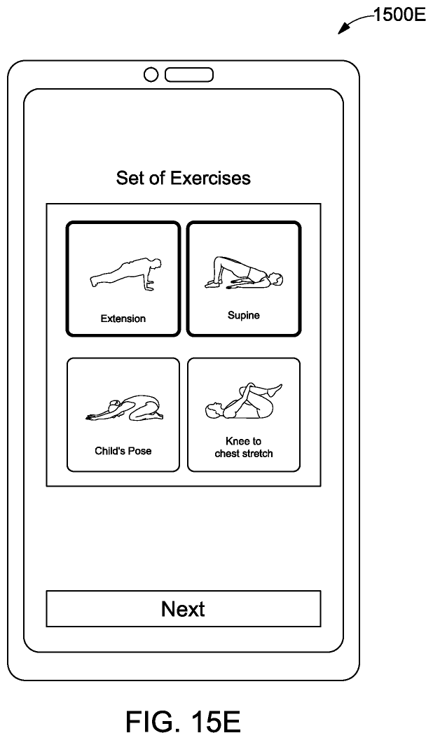

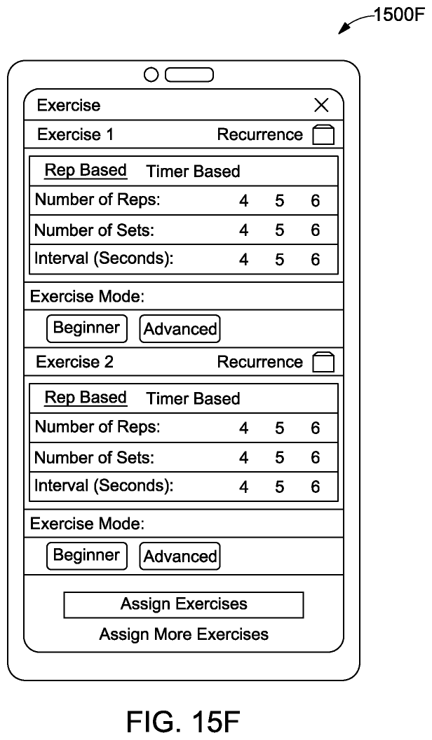

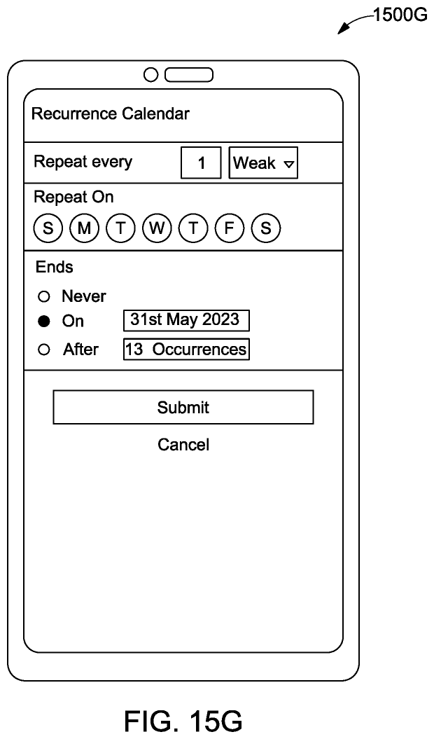

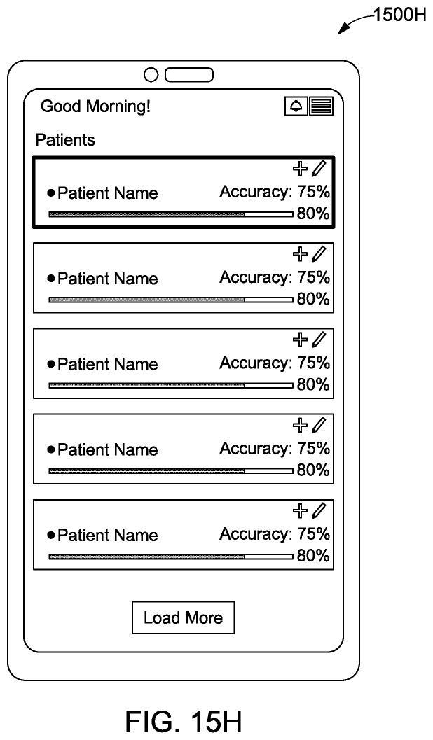

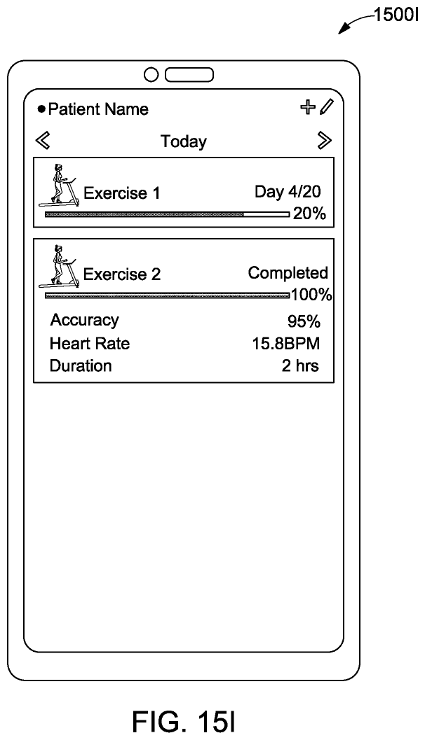

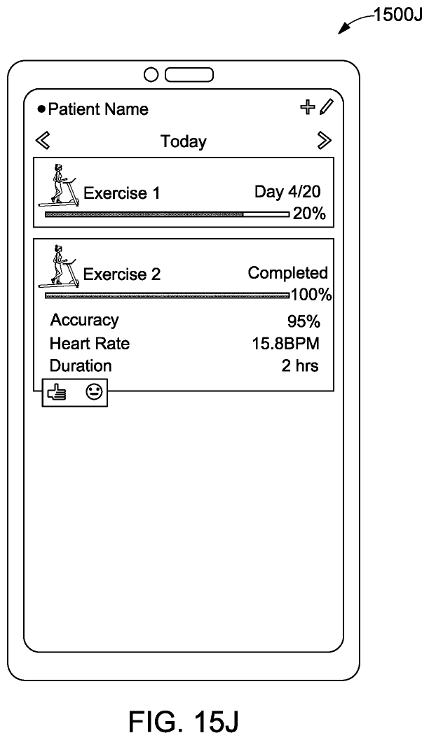

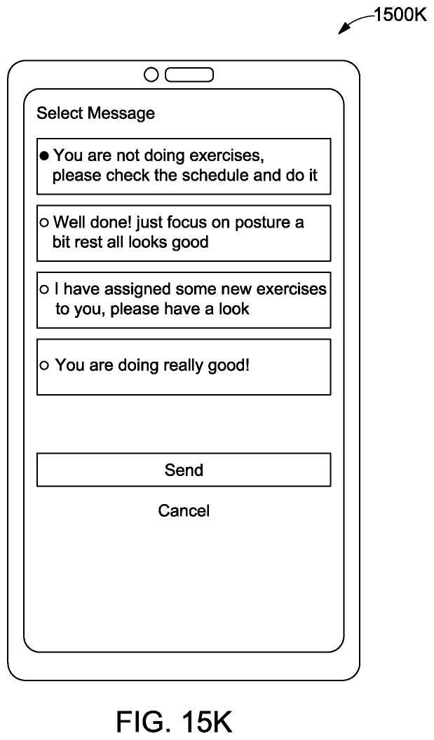

Upon downloading the application, the patient may register himself by providing his personal details, such as name, age, gender, email address, etc., and setting up a password for login. Once the patient registers himself, then the patient may login in the application using his login credentials, such as ‘username’ and ‘password’ as depicted via a GUI 900A. It should be noted that, if the patient forgets the password, then he can reset it using ‘forgot password link’. Upon login, the patient may select ‘a patient’ option from two options, i.e., ‘patient’, and ‘physio’ displayed to him, as depicted via a GUI 900B. A technique of accessing the application by the physiotherapist is further explained in detail in conjunction with FIGS. 15A-15K.

Upon login, the patient may select a symptom, e.g., ‘a back pain symptom’ from the list of symptoms being rendered to him via the GUI of the rendering device 110. Once the patient selects the appropriate symptom, the patient may provide his health records and the demographic data by scanning via the camera 110A, or the at least one camera 112. It should be noted that the patient may provide his health records and the demographic data by scanning via a camera of his smartphone.

The patient health records may include, for example, allergic reactions including drug allergies, chronic disease, family medical history, imaging reports (e.g., X-rays), medications and dosing, prescription record, surgeries and other procedures, list and dates of illness and hospitalizations, and the like. Further, examples of the demographic data of the patient may include, but are not limited to, name, age, gender, email address, date of birth, phone number, insurance information (e.g., insurance number), and the like. The patient's health record and the demographic data may be stored with the database 109 of the server 102.

Once the appropriate is selected, then the patient may be instructed to perform the at least one predefined movement. In continuation to the above example, the patient may be instructed to perform the at least one pre-defined movement to analyze current back pain condition of the patient. The at least one pre-defined movement for the back pain symptom may be, for example, ‘partial curl’. Further, while the patient might be performing the at least one pre-defined movement, the camera 110A or the at least one camera 112 may be configured to capture the first real-time video of the patient.

Further, the first real-time video may be processed to determine the set of health parameters, such as blood pressure, body temperature, pulse rate, or breathing rate of the patient while performing the at least one pre-defined movement, and movement or range of motion of body part requiring treatment, muscular strength, and the like. Based on the set of health parameters, the patient health record, and the demographic data, a set of exercises for the back pain treatment may be identified and rendered to the patient, as depicted via a GUI 900C. By way of example, the set of exercises may include five exercises, i.e., an extension exercise, a supine bridge exercise, a child's pose exercise, a knee to chest stretch exercise, and a knee rotation exercise.

Further, the patient may select one exercise from the five exercises rendered to him. For example, the patient may select 1st exercise, i.e., the extension exercise, as represented via a highlighted box in a GUI 900D. Once the patient selects the 1^{st} exercise, then the instructional video of the 1^{st} exercise may be rendered to the patient as depicted via a GUI 900E. Further, as depicted via the GUI 900E, the patient may be provided with an option ‘do not show again’, that the patient may select based on his requirement. For example, when the patient logs in the next day to perform exercise, he might not be interested in watching the instructional video again. In this case, the patient may select the provided option of ‘do not show again’.

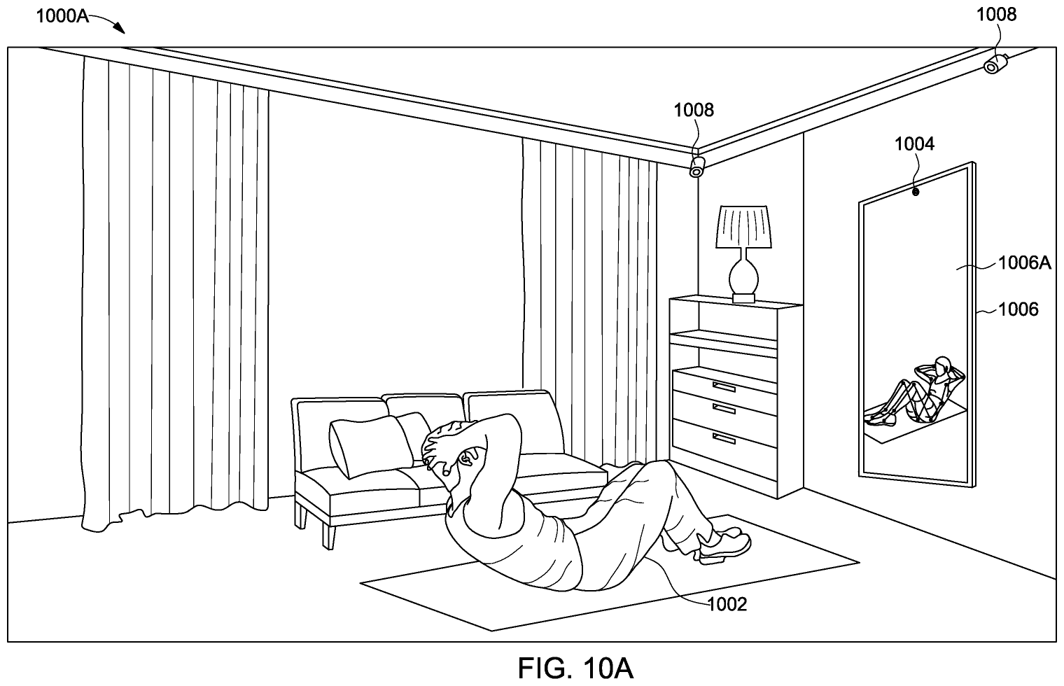

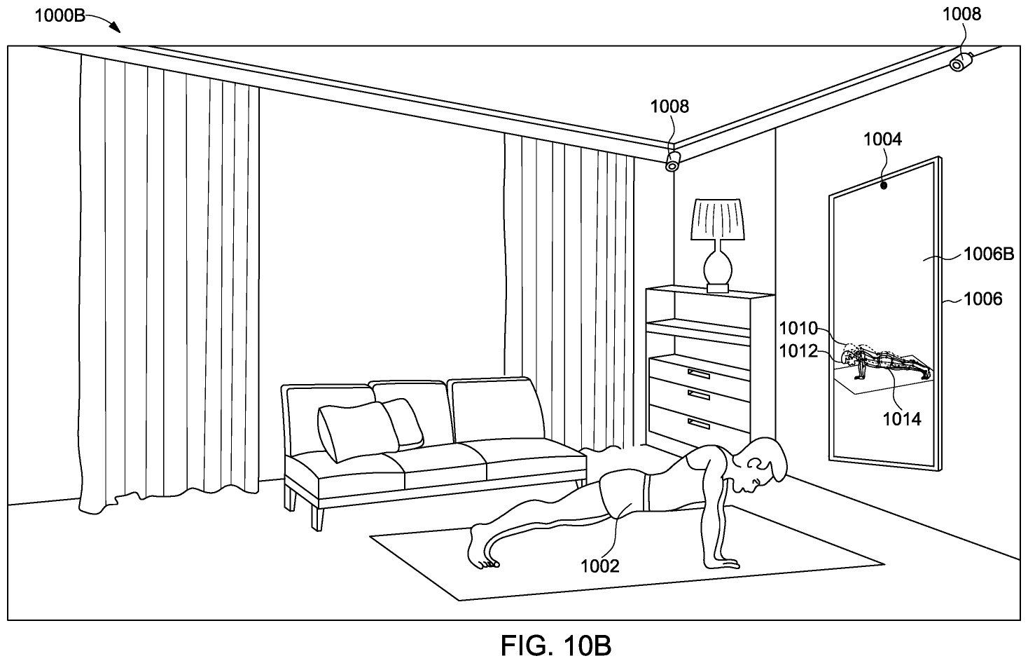

Referring now to FIGS. 10A and 10B, an exemplary scenario depicting a technique of capturing real-time videos of a patient is represented, in accordance with an exemplary embodiment. FIGS. 10A and 10B are explained in conjunction with FIGS. 1-9D. In the FIG. 10A, a patient 1002 performing the at least one pre-defined movement is depicted. The at least one pre-defined movement, for example, may be the partial curl. When the patient 1002 may be performing the partial curl, then, a camera 1004 of a rendering device 1006, i.e., the smart mirror may be configured to capture the first real-time video of the patient 1002, as depicted via a GUI 1006A of the rendering device 1006.

In some embodiment, the first real-time video may be captured via a set of cameras 1008 connected to the rendering device 1004 and a server (i.e., the server 102). It may be noted that each of the set of cameras 1008 may be positioned at center, along an edge, or at bottom of the rendering device 1004. With reference to FIG. 1, the rendering device 1006 may correspond to the rendering device 110. The camera 1004 may correspond to the camera 110A. Further, each of the set of cameras 1008 may correspond to the at least one camera 112.

Once the first real-time video is captured, the first real-time video may be transmitted to the first AI model 104 of the server 102. Further, the first AI model 104 may be configured to process the first real-time video to determine the current fitness state of the patient 1002. This has been already explained in detail in conjunction with FIGS. 1-9D. Once the current fitness state of the patient 1002 is determined, then the set of exercises may be identified for the back pain treatment. Further, the identified exercises may be rendered to the patient 1002 via the GUI 1006A of the rendering device 1006. With reference to FIG. 9C, the set of exercises rendered to the patient 1002 may correspond to the set of exercises rendered on the GUI 900C. By way of an example, the set of exercises may be extension exercise, supine bridge exercise, child's pose exercise, knee to chest stretch exercise, and knee rotation exercise. Further, the patient 1002 may select an exercise, for example, 1^{st} exercise, i.e., the extension exercise, as depicted via the GUI 900D of the FIG. 9. Upon selecting the exercise, the patient 1002 may have an option to view the instructional video of the extension exercise, as depicted via the GUI 900E of the FIG. 9E.

Further, when the patient 1002 starts performing the exercise, i.e., the extension exercise, the camera 1004 or the set of two cameras 1008 may be configured to capture the second-real-time video of the patient 1002, as depicted via the FIG. 10B. In particular, the camera 1004 may capture the second real-time video, while the patient may be performing the extension exercise, as depicted via a GUI 1006B of the rendering device 1006. As depicted via the GUI 1006B of the FIG. 10B, the rendering device 1006 shows a reflection 1010 of the patient 1002. The second real-time video may include a stream of poses and movements made by the patient 1002 to perform the extension exercise.

Further, with reference to FIG. 1, the second AI model 106 may be extracted to process the second real-time video in order to determine the deviation of the patient 1002 from a plurality of expected movements associated with the extension exercise. The deviation may be determined based on a target exercise performance 1012 of the healthy specimen. In order to determine the deviation, the second AI model may compare the set of patient mobility parameters of the patient 1002 with the set of target mobility parameters of the healthy specimen. The set of mobility parameters may be determined based on current exercise performance of the patient 1002. The method of determining the set of mobility parameters has been already covered in reference to FIGS. 1 and 2. Further, the comparison may be done based on the extension exercise performance of the healthy specimen.

In order to compare the set of mobility parameters of the patient 1002 with the set of target mobility parameters, the patient 1002 in the second real-time video may be overlayed with a pose skeleton model 1014. The pose skeletal model 1014 may include the plurality of key points based on the extension exercise. Further, each of the plurality of key points may be overlayed over the corresponding joint of the patient 1002 in the second real-time video. Additionally, the plurality of key points may be connected with lines representing bones of the patient to complete the pose skeletal model 1014.

In an embodiment, in order to compare the set of patient mobility parameters of the patient 1002 with the set of target mobility parameters, the reflection 1010 of the patient 1002 on the rendering device 1006 may be overlayed with one of the pose skeletal model 1014 and the plurality of key points overlayed on top of the reflection 1010, based on the current exercise performance and estimated future field of view and the current exercise performance and estimated future pose and motion of the patient 1002. Each of the plurality of key points is overlayed over a corresponding joint or a feature of the patient in the reflection 1010. Therefore, the rendering device shows the reflection 1010 of the current exercise performance of the patient.

The GUI 1006B of the rendering device 1006 shows the pose skeletal model 1014 overlayed on top of the reflection 1010 of the patient 1002, the target exercise performance 1012 of the exercise expert overlayed on the reflection 1010 of the patient 1002, the set of patient mobility parameters associated with the current exercise performance, and the set of target mobility parameters associated with the target exercise performance 1012. It may be noted that the pose skeletal model 1014 is automatically adjusted and normalized with respect to the reflection 1010 of the patient 1002 based on an estimated future distance of the patient relative to the rendering device 1006, the current exercise performance and estimated future field of view, and the current exercise performance and estimated future pose and motion of the patient. In some embodiments, transparency of the pose skeletal model 1014 may be adjustable by the patient 1002. In an embodiment, the pose skeletal model 1014 is completely transparent and invisible to the patient 1002. In such an embodiment, the pose skeletal model 1014 may be used by the second AI model 106 solely for computational purposes.

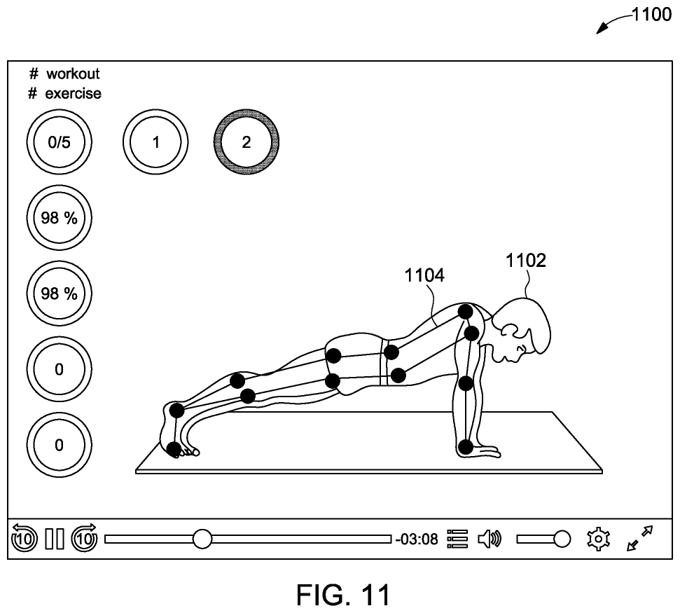

A technique of comparing the set of mobility parameters with the set of target mobility parameters is further explained in detail in conjunction with FIG. 11. Further, based on the comparison, the feedback may be rendered to the patient 1002. In present embodiment, the feedback may be a target exercise posture, i.e., the target exercise performance 1012 overlayed over the reflection 1010 of the patient 1002, as depicted via the GUI 1008B of the FIG. 10B.

Referring now to FIG. 11, a GUI 1100 displaying current exercise performance 1102 and pose skeletal model 1104 of the patient is represented, in accordance with an exemplary embodiment. FIG. 11 is explained in conjunction with FIGS. 1-10B. In an embodiment, the second AI model 106 may overlay the pose skeletal model 1104 (same as the pose skeleton model 1014) of the patient (i.e., the patient 1002) upon the second real-time video of the patient captured via the camera 1004 of by a rendering device (same as the rendering device 1002). Further, based on the overlaying, the second AI model 106 may determine the deviation of the patient 1002 from the plurality of expected movements associated with the extension exercise and render the at least one corrective action, i.e., the target exercise performance 1106. In some embodiment, the target exercise performance 1106 may not be overlayed and may be displayed near bottom right of display as depicted via the GUI 1100.

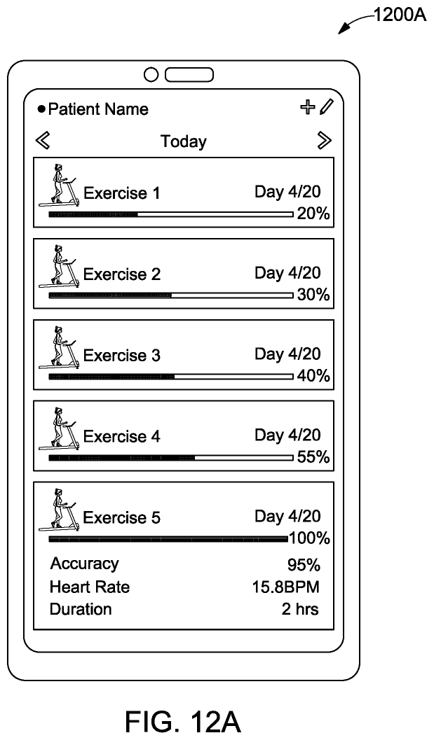

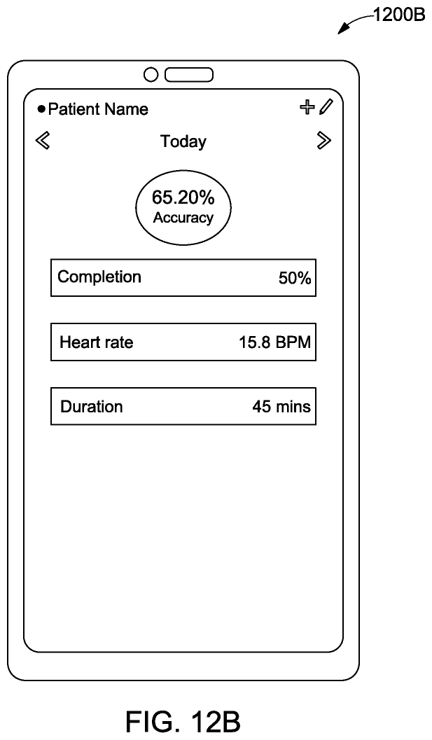

Referring now to FIGS. 12A-12B, GUIs depicting exercise reports generated based on assigned exercises performed by a patient are represented, in accordance with an exemplary embodiment. FIGS. 12A and 12B are explained in conjunction with the above FIGS. 1-11. It should be noted that, in addition to the rendered feedback, in some embodiment, the patient may be able to see the exercise reports generated for each exercise performed by the patient in a day. As will be appreciated, the exercise reports may be displayed to the patient (same as the patient 1002) via a GUI of his rendering device (i.e., the rendering device 1006). In some embodiment, the exercise reports may be displayed via the GUI of his smartphone communicatively coupled to the rendering device 1006.

For example, in one embodiment, the patient may be able to able to view completion status of each exercise performed by the patient in a day as depicted via a GUI 1200A of FIG. 12A. In continuation to the above example, where the set of five exercises are assigned to the patient for the back pain treatment, suppose for ‘20 days’. In this example, as depicted via the GUI 1200A, the patient may be able to see an exercise report of each of the set of five exercises. The exercise report may include completion status (in percentage) of each exercise of the set of five exercises. Additionally, the exercise report may include accuracy of each exercise, heart rate of the patient while the patient was performing each exercise, and duration of performing each exercise.

For example, suppose its fourth day on which the patient may have performed the set of five exercises. In this case, the patient may be rendered with the exercise report. In continuation to the above example, as depicted via the GUI 1200A, the exercise report for ‘exercise 5’, i.e., the knee rotation exercise, the completion status may be depicted as ‘100%’ or ‘complete’. Further, for other details, the patient may have selected ‘the exercise 5’, as depicted via a highlighted box. Upon selection, the patient may be able see the accuracy with which the patient may have performed the exercise 5, i.e., 95%. The heart rate of the patient while the patient was performing the exercise 5, i.e., 158 beats per minute (Bpm). The duration for which the exercise 5 was performed, i.e., 2 hours (hrs). In some embodiment, upon selecting an exercise of the set of five exercises, the exercise report for the exercise may be rendered to the patient, as depicted via a GUI 1200B of FIG. 12B.

In some embodiment, the exercise report may display improvement in the way of performing the exercise by the patient each time. By way of an example, the improvement in way of performing the exercise may be represented via a degree of movement done by the patient. In continuation to the above example, when the patient is performing the extension exercise, then in this case, during the first session, the patient may only be able to lift his body but may not be able to bend backwards. However, till 13th session, the patient may be able to bend backward for a few degrees (e.g., 40 degree). Then, in this case, the degree of movement, i.e., 40 degrees for the extension exercise may be rendered to the patient. In some embodiment, the exercise report may be rendered to the physiotherapist for evaluating progress in the patient's condition with respect to back pain condition.

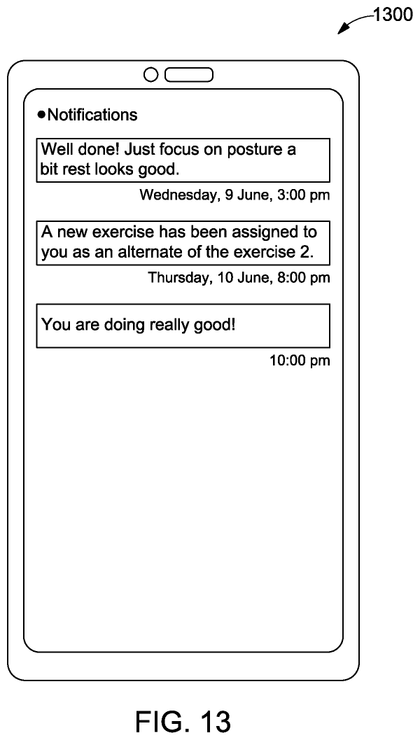

Referring now to FIG. 13, a GUI 1300 depicting notifications received by a patient based on assigned exercises is represented, in accordance with an exemplary embodiment. FIG. 13 is explained in conjunction with FIGS. 1-12B.

In continuation to the above example, when the patient is assigned the set of five exercises for his back pain treatment, the patient may be receiving the notifications based on daily performance. The notifications, for example, may include the feedback of the exercise being performed by the patient, the reminder received upon identification of the failure in completion of an exercise by the patient, the alternative exercise suggested to the patient. By way of an example, when the patient may be performing the exercise, for example, exercise 4 (i.e., the knee to chest exercise), then based on processing of a corresponding second real-time video and the comparison, the feedback may be generated and rendered to the patient. For example, the feedback may be, ‘well done, just focus on posture a little bit rest all looks good’, depicted as a second notification via the GUI 1300.

By way of another example, upon determining the failure in completion of the exercise for the pre-defined time interval, the reminder may be sent to the patient. In continuation to the above example, when the patient may not have performed the set of exercises for a consecutive 5 days (i.e., the pre-defined time interval). Then, the reminder may be sent to the patient daily, until the patient resumes performing the set of exercises assigned to him for the back pain treatment. By way of example, the reminder may be ‘you are not doing exercises, please check your exercise schedule and do it’, depicted as a first notification via the GUI 1300.

By way of yet another example, when from the set of five exercises assigned to the patient, the patient may not have done one exercise (for example, ‘exercise 2’) for consecutive 3 days. Then, the alternative exercises may be suggested to him as the replacement of the ‘exercise 2’. Further, based on the suggested exercise, a notification, e.g., ‘A new exercise has been assigned to you as an alternate of the ‘exercise 2’’ may be rendered to the patient. By way of yet another example, when the patient may have done each of the set of exercises really well for a day. Then, for that day, a notification, ‘you are doing really good’ may be rendered as the feedback to the patient. As will be appreciated, each notification may be generated by the server 102 based on the real-time processing of the second real-time video that is captured by the second AI model 106.

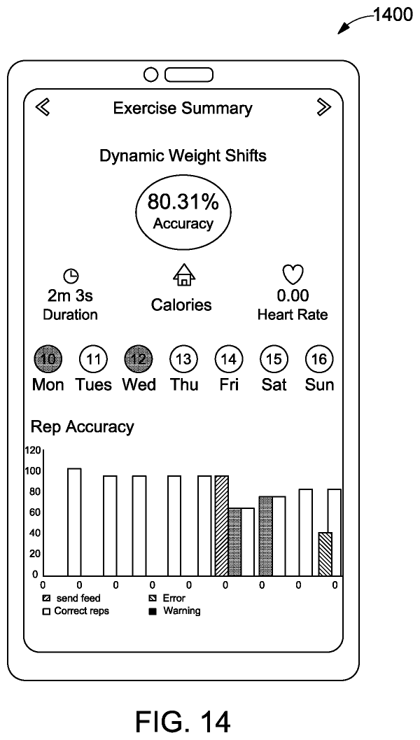

Referring now to FIG. 14, a GUI 1400 depicting a summarized report generated based on monitoring a patient is represented, in accordance with an exemplary embodiment. FIG. 14 is explained in conjunction with FIGS. 1-13. As will be appreciated, the summarized report may be generated by the second AI model 106. In order to generate the summarized report, the second AI model 106 may be configured to monitor each of the set of exercises performed by the patient based on the corresponding second real-time video. In continuation to the above example, consider a scenario where 20 sessions (i.e., for 20 days) of the set of five exercises was assigned to the patient for the back pain treatment.

In this scenario, every day when the patient may be performing the exercise in front of the rendering device 110, then the camera 110A of the rendering device 110 or the at least one camera 112 may capture and send the second real-time video of each day of each exercise to the second AI model 106. Further, based on the processing, the second AI model 106 may generate the summarized report as depicted via the GUI 1400. As depicted via the GUI 1400, the patient may be able to view his performance of each day as a graphical representation. In addition, the patient may be able to see his accuracy of completion of each of the 20 sessions, along with duration, calories burnt, and heart rate of the patient.

Referring now to FIGS. 15A-15K, an exemplary technique of assisting a physiotherapist for providing remote physiotherapy sessions to a patient is depicted, in accordance with an exemplary embodiment. FIG. 15 is explained in conjunction with FIGS. 1-14. As discussed in the above FIG. 1, in some embodiment, the server 102 may be configured to assist the physiotherapist in providing the remote physiotherapy sessions to the patient. In such an embodiment, in order to assist the physiotherapist, initially, the physiotherapist may connect to the server 102 by downloading and registering with the associated application. Upon registering, every time the physiotherapist wants to connect to the server, the physiotherapist may login to the application using his associated credentials as depicted via a GUI 1500A of FIG. 15A.

Upon login, the physiotherapist may select ‘a physio’ option from two options, i.e., ‘patient’, and ‘physio’ displayed to him, as depicted via a GUI 1500B of FIG. 15B. Once the physiotherapist logs in, the physiotherapist may be able to see a list of patients to whom he is providing the remote physiotherapy sessions. The list of patients may include, patient's name and overall accuracy of each session performed until now, as depicted via a GUI 1500C in FIG. 15C. In continuation to the above example, 20 sessions of each of the set of five exercises were assigned to the patient. In this example, if the patient has taken 13 sessions out of 14 sessions that happened until the current date, then the accuracy for that patient may be rendered to the physiotherapist. As will be appreciated, the accuracy may be determined by the second AI model 106 based on monitoring of the patient using the corresponding second real-time video.

Further, consider an example, when a new patient, e.g., patient A, may be interested in taking treatment for the back pain treatment by the physiotherapist, as depicted via a highlighted box in a GUI 1500D of FIG. 15D. Since the patient A is the new patient and a set of exercises that need to be assigned to the patient A are not yet identified, in this case, the accuracy may not be presented as depicted via the GUI 1500D. Further, upon receiving a request from the patient A for the remote physiotherapy sessions, the first AI model 104 may be configured to receive and analyze information (i.e., the first real-time video, the patient health record data, and the demographic data) to identifying the set of exercises for the patient. The identified set of exercises, for example, a set of four exercises, may be presented to the physiotherapist as depicted via a GUI 1500E in FIG. 15E. Further, from the set of four exercises, the physiotherapist may select one or more exercises from the set of four exercises identified by the first AI model 104, based on his analysis. For example, the physiotherapist may select two exercises, e.g., exercise 1, exercise 2 from the set of four exercises as depicted via a highlighted box in the GUI 1500E.

In addition to selection of the two exercises, the physiotherapist may be able to define the number of repetitions and the number of sets for each exercise, i.e., the exercise 1 and exercise 2, as depicted via a GUI 1500F of FIG. 15F. Further, the physiotherapist may be able to define a time interval (in seconds) for each repetition and each set of each exercise, as depicted via the GUI 1500F. Furthermore, the physiotherapist may be able to select one of the plurality of modes for each exercise as depicted via the GUI 1500F.

Additionally, the second AI model 106 may assist the physiotherapist in defining a number of sessions required for the back pain treatment by rendering a GUI 1500G of FIG. 15G. The GUI 1500G represents a recurrence calendar that may be rendered to the physiotherapist. As depicted via the GUI 1500G, the physiotherapist may select time period after which each of the two exercises needs to be repeated, for example, ‘repeat every-1 week’. Further, the physiotherapist may select a day on which the two exercises need to be repeated. Furthermore, the physiotherapist may select a time interval after which the treatment of back pain for the patient may end. As depicted via the GUI 1500G, the physiotherapist may select one or more options from a set of options, i.e., never, end date, and number of occurrences (i.e., sessions), based on the current fitness state of the patient determined by the first AI model 104. For example, the physiotherapist may select the end date as: 31 Dec. 2023, and the number of occurrences as 13 occurrences.

Further, the physiotherapist may be able to see accuracy of a set of exercises assigned to each of the plurality of patients to whom he is providing treatment, as depicted via a GUI 1500H of FIG. 15H. The physiotherapist may select any patient to view progress details of the patient, such as, completion of exercise, duration for which each assigned exercise is performed, and the like. As depicted via a highlighted box in the GUI 150HI, the physiotherapist may have selected ‘the patient A’.

Upon selecting ‘the patient A’, the physiotherapist may be able to see an exercise report of an exercise from the two exercises. The exercise report may include completion status (in percentage) of each exercise of the set of five exercises. Additionally, the exercise report may include accuracy of each exercise, heart rate of the patient while the patient was performing each exercise, and duration of performing each exercise. For example, the physiotherapist may select the exercise 2. Upon selecting the exercise 2, the physiotherapist may be able to see the exercise report of the exercise 2, as depicted via a GUI 1500I of FIG. 15I.

Further, in addition to the feedback generated by the second AI model 106, the second AI model 106 may assist the physiotherapist to provide the feedback on each exercise assigned to the patient. As depicted via a GUI 1500J in FIG. 15J, the physiotherapist may provide the feedback for each exercise (e.g., the exercise 2) by selecting an emoticon icon from a list of emoticon icons rendered by the second AI model 106 to the physiotherapist. In addition, the second AI model 106 may render a list of messages to the physiotherapist. The physiotherapist may select a message, e.g., ‘you are not doing exercises, please check schedule and do it’ from the list of messages to provide the feedback to the patient, as depicted via a GUI 15K of FIG. 15K.

As will be appreciated, the technique of assisting the physiotherapist for providing remote physiotherapy sessions to the patient is just one exemplary embodiment. However, as already discussed the above FIGS. 1-14, the technique of providing the remote physiotherapy sessions to patients is automatically executed by the first AI model 104 and the second AI model 106 of the server 102.

Some embodiments of the present disclosure may be employed in a gymnasium, rehab, dance studios, theatre, or any other use case scenario. The gymnasium may include, for example, multiple exercise machines and equipment for performing multiple activities by a user. The user may use a rendering device (for example, the rendering device 110) to receive remote workout assistance. The camera of the rendering device or at least one camera may capture real-time videos of the user and render feedback to the user for improvising the activities being performed. Further, the rendering device 110 may be configured to provide the audio feedback via a Bluetooth headset or a speaker.

Some embodiments of the present disclosure may be implemented as an AI-based health and fitness system training method. The method includes capturing a first real-time video of a user using a camera, processing the first real-time video of the patient to determine a set of health parameters, analyzing the set of health parameters and at least one of patient health records and demographic data to determine a current fitness state of the patient, identifying a set of exercises to be performed by the user, etc.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

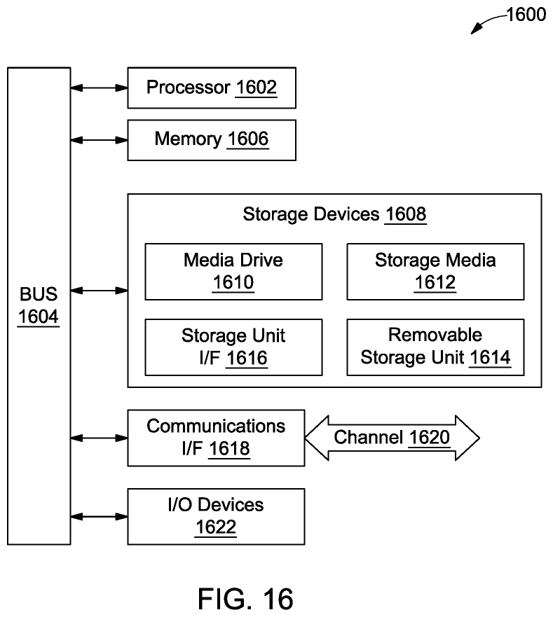

The disclosed methods and systems may be implemented on a conventional or a general-purpose computer system, such as a personal computer (PC) or server computer. Referring now to FIG. 16, an exemplary computing system 1600 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 1600 may represent, for example, a user device such as a desktop, a laptop, a mobile phone, personal entertainment device, DVR, and so on, or any other type of special or general-purpose computing device as may be desirable or appropriate for a given application or environment. The computing system 1600 may include one or more processors, such as a processor 1602 that may be implemented using a general or special purpose processing engine such as, for example, a microprocessor, microcontroller or other control logic. In this example, the processor 1602 is connected to a bus 1604 or other communication medium. In some embodiments, the processor 1602 may be an Artificial Intelligence (AI) processor, which may be implemented as a Tensor Processing Unit (TPU), or a graphical processor unit, or a custom programmable solution Field-Programmable Gate Array (FPGA).

The computing system 1600 may also include a memory 1606 (main memory), for example, Random Access Memory (RAM) or other dynamic memory, for storing information and instructions to be executed by the processor 1602. The memory 1606 also may be used for storing temporary variables or other intermediate information during execution of instructions to be executed by the processor 1602. The computing system 1600 may likewise include a read only memory (“ROM”) or other static storage device coupled to the bus 1604 for storing static information and instructions for the processor 1602.

The computing system 1600 may also include storage devices 1608, which may include, for example, a media drive 1610 and a removable storage interface. The media drive 1610 may include a drive or other mechanism to support fixed or removable storage media, such as a hard disk drive, a floppy disk drive, a magnetic tape drive, an SD card port, a USB port, a micro-USB, an optical disk drive, a CD or DVD drive (R or RW), or other removable or fixed media drive. A storage media 1612 may include, for example, a hard disk, magnetic tape, flash drive, or other fixed or removable medium that is read by and written to by the media drive 1610. As these examples illustrate, the storage media 1612 may include a computer-readable storage medium having stored therein particular computer software or data.

In alternative embodiments, the storage devices 1608 may include other similar instrumentalities for allowing computer programs or other instructions or data to be loaded into the computing system 1600. Such instrumentalities may include, for example, a removable storage unit 1614 and a storage unit interface 1616, such as a program cartridge and cartridge interface, a removable memory (for example, a flash memory or other removable memory module) and memory slot, and other removable storage units and interfaces that allow software and data to be transferred from the removable storage unit 1614 to the computing system 1600.

The computing system 1600 may also include a communications interface 1618. The communications interface 1618 may be used to allow software and data to be transferred between the computing system 1600 and external devices. Examples of the communications interface 1618 may include a network interface (such as an Ethernet or other NIC card), a communications port (such as for example, a USB port, a micro-USB port), Near field Communication (NFC), etc. Software and data transferred via the communications interface 1618 are in the form of signals which may be electronic, electromagnetic, optical, or other signals capable of being received by the communications interface 1618. These signals are provided to the communications interface 1618 via a channel 1620. The channel 1620 may carry signals and may be implemented using a wireless medium, wire or cable, fiber optics, or other communications medium. Some examples of the channel 1620 may include a phone line, a cellular phone link, an RF link, a Bluetooth link, a network interface, a local or wide area network, and other communications channels.

The computing system 1600 may further include Input/Output (I/O) devices 1622. Examples may include, but are not limited to a display, keypad, microphone, audio speakers, vibrating motor, LED lights, etc. The I/O devices 1622 may receive input from a user and also display an output of the computation performed by the processor 1602. In this document, the terms “computer program product” and “computer-readable medium” may be used generally to refer to media such as, for example, the memory 1606, the storage devices 1608, the removable storage unit 1614, or signal(s) on the channel 1620. These and other forms of computer-readable media may be involved in providing one or more sequences of one or more instructions to the processor 1602 for execution. Such instructions, generally referred to as “computer program code” (which may be grouped in the form of computer programs or other groupings), when executed, enable the computing system 1600 to perform features or functions of embodiments of the present invention.

In an embodiment where the elements are implemented using software, the software may be stored in a computer-readable medium and loaded into the computing system 1600 using, for example, the removable storage unit 1614, the media drive 1610 or the communications interface 1618. The control logic (in this example, software instructions or computer program code), when executed by the processor 1602, causes the processor 1602 to perform the functions of the invention as described herein.

As will be appreciated by those skilled in the art, the techniques described in the various embodiments discussed above are not routine, or conventional, or well understood in the art. The techniques discussed above provide for remote physiotherapy sessions. The techniques first capture, via at least one camera, a first real-time video of a patient performing at least one predefined movement. The techniques may then process in real-time, via a first Artificial Intelligence (AI) model, the real-time video of the patient to determine a set of health parameters based on the at least one predefined movement performed by the patient. The techniques may then analyze, via the first AI model, the set of health parameters and at least one of patient health records and demographic data to determine a current fitness state of the patient. The techniques may then identify, via the first AI model, a set of exercises to be performed by the patient, based on the current fitness state of the patient. The techniques may then capture, via the at least one camera, a second real-time video of the patient performing an exercise from the set of exercises. The real-time video may include a stream of poses and movements made by the patient to perform the exercise. The techniques may then extract a second AI model based on the current fitness state of the patient and the exercise being performed by the patient. The second AI model may be configured to determine a deviation of the patient from a plurality of expected movements associated with the exercise based on target exercise performance of a healthy specimen. The techniques may then process in real-time, the second real-time video of the patient by the second AI model to determine a set of patient mobility parameters based on current exercise performance of the patient. The techniques may then compare the set of patient mobility parameters with a set of target mobility parameters by the second AI model. The set of target mobility parameters may correspond to the healthy specimen. The techniques may then generate, via the second AI model, feedback for the patient based on comparison of the set of patient mobility parameters with the set of target mobility parameters. The feedback may include at least one of corrective actions or alerts. The feedback may be at least one of visual feedback, aural feedback, or haptic feedback. The techniques may then render, via the second AI model, the feedback on a rendering device.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described a method and system for providing remote physiotherapy sessions. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for providing remote physiotherapy sessions, the method comprising:
capturing, by at least one camera, a first real-time video of a patient performing at least one predefined movement;
processing in real-time, by a first Artificial Intelligence (AI) model, the first real-time video of the patient to determine a set of health parameters based on the at least one predefined movement performed by the patient;
analyzing, by the first AI model, the set of health parameters and at least one of patient health records and demographic data to determine a current fitness state of the patient;
identifying, by the first AI model, a set of exercises to be performed by the patient, based on the current fitness state of the patient;
capturing, by the at least one camera, a second real-time video of the patient performing an exercise from the set of exercises, wherein the second real-time video comprises a stream of poses and movements made by the patient to perform the exercise;
extracting a second AI model based on the current fitness state of the patient and the exercise being performed by the patient, wherein the second AI model is configured to determine a deviation of the patient from a plurality of expected movements associated with the exercise based on target exercise performance of a healthy specimen;
processing in real-time, by the second AI model, the second real-time video of the patient to determine a set of patient mobility parameters based on current exercise performance of the patient;
comparing, by the second AI model, the set of patient mobility parameters with a set of target mobility parameters, wherein the set of target mobility parameters corresponds to the healthy specimen;
generating, by the second AI model, feedback for the patient based on comparison of the set of patient mobility parameters with the set of target mobility parameters, wherein the feedback comprises at least one of corrective actions or alerts, and wherein the feedback comprises at least one of visual feedback, aural feedback, or haptic feedback; and
rendering, by the second AI model, the feedback on a rendering device.

2. The method of claim 1, further comprising overlaying, by the second AI model, the patient in the second real-time video with a pose skeletal model, wherein the pose skeletal model comprises a plurality of key points based on the exercise, and wherein each of the plurality of key points is overlayed over a corresponding joint of the patient in the second real-time video.

3. The method of claim 2, wherein rendering the feedback comprises:
overlaying one of at least one corrective action over the pose skeletal model overlayed on the second real-time video of the patient;
displaying the alerts on a Graphical User Interface (GUI) of the rendering device; and
outputting the aural feedback to the patient, via a speaker.

4. The method of claim 3, wherein the feedback comprises generating a warning to the patient comprising:
indication for correcting a current pose of the patient; and
indication for correcting motion associated with the current pose of the patient.

5. The method of claim 1, further comprising:
rendering, via the GUI, the set of exercises to the patient; and
receiving, via the GUI, the exercise as patient selection.

6. The method of claim 1, further comprising customizing, by the second AI model, the exercise for the patient, based on comparison of the set of patient mobility parameters with the set of target mobility parameters, wherein customizing the exercise comprises:
defining a number of repetitions and a number of sets of the exercise for the patient; and
selecting one of a plurality of modes for the exercise, based on the current fitness state of the patient.

7. The method of claim 1, further comprising:
identifying, by the second AI model, a failure in completion of the exercise by the patient; and
sending a reminder to the patient after expiry of a pre-defined time interval for completing of the exercise, in response to identifying the failure in completion of the exercise by the patient.

8. The method of claim 7, further comprising:
suggesting, by the second AI model, an alternative exercise instead of the exercise, in response to identified repeated failures in completion of the exercise by the patient.

9. The method of claim 1, further comprising:
monitoring, by the second AI model, each of the set of exercises being performed by the patient based on a corresponding second real-time video of the patient;
generating, by the second AI model, a summarized report corresponding to the patient based on the monitoring; and
rendering, via the GUI, the summarized report to the patient.

10. The method of claim 9, further comprising:
validating patient performance based on the summarized report; and
providing an authorization to the patient to perform one or more action, upon a successful validation.

11. A system for providing remote physiotherapy sessions, the system comprising:
a processor; and
a memory coupled to the processor, wherein the memory stores processor executable instructions, which, on execution, causes the processor to:
capture, by at least one camera, a first real-time video of a patient performing at least one predefined movement;
process in real-time, by a first Artificial Intelligence (AI) model, the first real-time video of the patient to determine a set of health parameters based on the at least one predefined movement performed by the patient;
analyze, by the first AI model, the set of health parameters and at least one of patient health records and demographic data to determine a current fitness state of the patient;
identify, by the first AI model, a set of exercises to be performed by the patient, based on the current fitness state of the patient;
capture, by the at least one camera, a second real-time video of the patient performing an exercise from the set of exercises, wherein the second the real-time video comprises a stream of poses and movements made by the patient to perform the exercise;
extract a second AI model based on the current fitness state of the patient and the exercise being performed by the patient, wherein the second AI model is configured to determine a deviation of the patient from a plurality of expected movements associated with the exercise based on target exercise performance of a healthy specimen;
process in real-time, by the second AI model, the second real-time video of the patient to determine a set of patient mobility parameters based on current exercise performance of the patient;
compare, by the second AI model, the set of patient mobility parameters with a set of target mobility parameters, wherein the set of target mobility parameters corresponds to the healthy specimen;
generate, by the second AI model, feedback for the patient based on comparison of the set of patient mobility parameters with the set of target mobility parameters, wherein the feedback comprises at least one of corrective actions or alerts, and wherein the feedback comprises at least one of visual feedback, aural feedback, or haptic feedback; and
render, by the second AI model, the feedback on a rendering device.

12. The system of claim 11, wherein the processor executable instructions further cause the processor to:
overlay, by the second AI model, the patient in the second real-time video with a pose skeletal model, wherein the pose skeletal model comprises a plurality of key points based on the exercise, and wherein each of the plurality of key points is overlayed over a corresponding joint of the patient in the second real-time video.

13. The system of claim 12, wherein, to render the feedback, the processor executable instructions further cause the processor to:
overlay one of at least one corrective action over the pose skeletal model overlayed on the second real-time video of the patient;
display the alerts on a Graphical User Interface (GUI) of the rendering device; and
output the aural feedback to the patient, via a speaker.

14. The system of claim 13, wherein the feedback comprises generating a warning to the patient comprising:
indication for correcting a current pose of the patient; and
indication for correcting motion associated with the current pose of the patient.

15. The system of claim 11, wherein the processor executable instructions further cause the processor to:
rendering, via the GUI, the set of exercises to the patient; and
receiving, via the GUI, the exercise as patient selection.

16. The system of claim 11, wherein the processor executable instructions further cause the processor to customize, by the second AI model, the exercise for the patient, based on comparison of the set of patient mobility parameters with the set of target mobility parameters, and wherein, to customize the exercise, the processor executable instructions further cause the processor to:
define a number of repetitions and a number of sets of the exercise for the patient; and
select one of a plurality of modes for the exercise, based on the current fitness state of the patient.

17. The system of claim 11, wherein the processor executable instructions further cause the processor to:
identify, by the second AI model, a failure in completion of the exercise by the patient; and
send a reminder to the patient after expiry of a pre-defined time interval for completing of the exercise, in response to identifying the failure in completion of the exercise by the patient.

18. The system of claim 17, wherein the processor executable instructions further cause the processor to:
suggest, by the second AI model an alternative exercise instead of the exercise, in response to identified repeated failures in completion of the exercise by the patient.

19. The system of claim 11, wherein the processor executable instructions further cause the processor to:
monitor, by the second AI model, each of the set of exercises being performed by the patient based on a corresponding second real-time video of the patient;
generate, by the second AI model, a summarized report corresponding to the patient based on the monitoring;
render, via the GUI, the summarized report to the patient;
validate patient performance based on the summarized report; and
provide an authorization to the patient to perform one or more actions, upon a successful validation.

20. A non-transitory computer-readable medium storing computer-executable instructions for providing remote physiotherapy sessions, the stored instructions, when executed by a processor, cause the processor to perform operations comprises:
capturing, by at least one camera, a first real-time video of a patient performing at least one predefined movement;
processing in real-time, by a first Artificial Intelligence (AI) model, the first real-time video of the patient to determine a set of health parameters based on the at least one predefined movement performed by the patient;
analyzing, by the first AI model, the set of health parameters and at least one of patient health records and demographic data to determine a current fitness state of the patient;
identifying, by the first AI model, a set of exercises to be performed by the patient, based on the current fitness state of the patient;
capturing, by the at least one camera, a second real-time video of the patient performing an exercise from the set of exercises, wherein the second the real-time video comprises a stream of poses and movements made by the patient to perform the exercise;
extracting a second AI model based on the current fitness state of the patient and the exercise being performed by the patient, wherein the second AI model is configured to determine a deviation of the patient from a plurality of expected movements associated with the exercise based on target exercise performance of a healthy specimen;
processing in real-time, by the second AI model, the second real-time video of the patient to determine a set of patient mobility parameters based on current exercise performance of the patient;
comparing, by the second AI model, the set of patient mobility parameters with a set of target mobility parameters, wherein the set of target mobility parameters corresponds to the healthy specimen;
generating, by the second AI model, feedback for the patient based on comparison of the set of patient mobility parameters with the set of target mobility parameters, wherein the feedback comprises at least one of corrective actions or alerts, and wherein the feedback comprises at least one of visual feedback, aural feedback, or haptic feedback; and
rendering, by the second AI model, the feedback on a rendering device.
